(19) United States
(12) Patent Application Publication
(10) Pub. No.: US 2024/0129451 A1
(43) Pub. Date: Apr. 18, 2024

(54) EXTENDED REALITY HEADSET CAMERA SYSTEM FOR COMPUTER ASSISTED NAVIGATION IN SURGERY

(71) Applicant: GLOBUS MEDICAL, INC., AUDUBON, PA (US)

(72) Inventors: Weston Healy, Durham, NC (US); Thomas Calloway, Pelham, NH (US); Norbert Johnson, North Andover, MA (US); Keerthighaan Kanagasegar, Norristown, PA (US)

(21) Appl. No.: 18/483,105

(22) Filed: Oct. 9, 2023

Related U.S. Application Data
(63) Continuation of application No. 17/478,201, filed on Sep. 17, 2021, now Pat. No. 11,838,493.
Application No. 17/478,201 is a continuation of application No. 16/869,735, filed on May 8, 2020, now Pat. No. 11,153,555.

(51) Int. Cl. H04N 13/344; A61B 34/20; H04N 5/33; H04N 13/239; H04N 23/12

(52) U.S. Cl. CPC H04N 13/344; A61B 34/20; H04N 5/33; H04N 13/239; H04N 23/12; A61B 2034/2057; A61B 2034/2072; A61B 34/30

(57) ABSTRACT

A head-mounted extended reality (XR) display device includes a rigid mounting element coupled to a frame. The XR display device further includes right-side and left-side visible light cameras coupled to the rigid mounting element, right-side and left-side near-infrared (NIR) cameras coupled to the rigid mounting element, and an NIR light-emitting diode (LED) configured to illuminate a region within a field of view of the NIR cameras. The visible light cameras are configured to capture stereoscopic visible light images within a field of view of the user when the user is wearing the frame, and the NIR cameras are configured to capture stereoscopic NIR images within the field of view of the user when the user is wearing the frame.

### CROSS-REFERENCE TO RELATED APPLICATIONS

This application is a continuation of U.S. patent application Ser. No. 17/478,201 filed on Sep. 17, 2021, which is a continuation application of U.S. patent application Ser. No. 16/869,735, filed on May 8, 2020, which is incorporated by reference herein in its entirety for all purposes.

### FIELD

The present disclosure relates to medical devices and systems, and more particularly, computer assisted navigation in surgery using an extended reality headset camera system.

### BACKGROUND

Computer assisted navigation in surgery provides surgeons with enhanced visualization of surgical instruments with respect to radiographic images of the patient's anatomy. However, existing navigation systems have limitations on usability and ergonomics for the surgeon, including 1) necessitating that the surgeon turn his/her head away from the patient and surgical instrument to view navigation information, 2) imposing reliance on other personnel to operate software functions of various equipment in the operating room, and 3) intermittent pauses with providing navigation during surgery while personnel and/or objects obstruct the ability of a tracking component to track poses of the patient, the robot, and surgical instruments.

### SUMMARY

Various embodiments disclosed herein are directed to improvements in computer assisted navigation during surgery. An extended reality (XR) headset is operatively connected to the surgical system to provide an interactive environment through which a surgeon, assistant, and/or other personnel can view and manipulate patient images, view and manipulate computer generated navigation information, and/or control surgical equipment in an operating room.

Some embodiments of the present disclosure are directed to a head-mounted extended reality (XR) display device. The XR display device includes a frame configured to be worn by a user's head and a rigid mounting element coupled to the frame. The XR display device further includes a right-side visible light camera coupled to the rigid mounting element, and a left-side visible light camera coupled to the rigid mounting element. The XR display device further includes a right-side near-infrared (NIR) camera coupled to the rigid mounting element, a left-side NIR camera coupled to the rigid mounting element, and at least one NIR light-emitting diode (LED) configured to illuminate a region within a field of view of the right-side NIR camera and the left-side NIR camera. The right-side visible light camera and the left-side visible light camera are configured to capture stereoscopic visible light images within a field of view of the user when the user is wearing the frame. The right-side NIR camera and the left-side NIR camera are configured to capture stereoscopic NIR images within the field of view of the user when the user is wearing the frame.

Some embodiments of the present disclosure are directed to an XR camera assembly for a head-mounted display device. The XR camera assembly includes a rigid mounting element configured to be coupled to a head-wearable frame. The XR camera assembly further includes a right-side visible light camera coupled to the rigid mounting element, and a left-side visible light camera coupled to the rigid mounting element. The XR camera assembly further includes a right-side near-infrared (NIR) camera coupled to the rigid mounting element, a left-side NIR camera coupled to the rigid mounting element, and at least one NIR light-emitting diode (LED) configured to illuminate a region within a field of view of the right-side NIR camera and the left-side NIR camera. The right-side visible light camera and the left-side visible light camera are configured to capture stereoscopic visible light images within a field of view of the user when the user is wearing the frame. The right-side NIR camera and the left-side NIR camera are configured to capture stereoscopic NIR images within the field of view of the user when the user is wearing the frame.

Some embodiments of the present disclosure are directed to a method of generating extended reality (XR) imagery. The method includes capturing a first plurality of visible light stereoscopic images of a scene within a field of view of a pair of visible light cameras coupled to a rigid mounting element during a time period. The method further includes capturing a second plurality of near-infrared (NIR) stereoscopic images of a scene within a field of view of a pair of NIR cameras coupled to the rigid mounting element during the time period. The method further includes determining a location of a first object in the first plurality of stereoscopic images within a three-dimensional space. The method further includes determining a location of a second object in the second plurality of stereoscopic images within the three-dimensional space.

Other surgical systems, XR headsets, components, and corresponding methods and computer program products according to embodiments will be or become apparent to one with skill in the art upon review of the following drawings and detailed description. It is intended that all such surgical systems, XR headsets, components, and corresponding methods and computer program products be included within this description, be within the scope of the present disclosure, and be protected by the accompanying claims. Moreover, it is intended that all embodiments disclosed herein can be implemented separately or combined in any way and/or combination.

### BRIEF DESCRIPTION OF THE DRAWINGS

The accompanying drawings, which are included to provide a further understanding of the disclosure and are incorporated in a constitute a part of this application, illustrate certain non-limiting embodiments of inventive concepts. In the drawings:

FIG. 1 illustrates an embodiment of a surgical system according to some embodiments of the present disclosure;

FIG. 2 illustrates a surgical robot component of the surgical system of FIG. 1 according to some embodiments of the present disclosure;

FIG. 3A illustrates a camera tracking system component of the surgical system of FIG. 1 according to some embodiments of the present disclosure;

FIGS. 3B and 3C illustrate a front view and isometric view of another camera tracking system component which may be used with the surgical system of FIG. 1 according to some embodiments of the present disclosure;

FIG. 4 illustrates an embodiment of an end effector that is connectable to a robot arm and configured according to some embodiments of the present disclosure;

FIG. 5 illustrates a medical operation in which a surgical robot and a camera system are disposed around a patient;

FIG. 6 illustrates a block diagram view of the components of the surgical system of FIG. 5 being used for a medical operation;

FIG. 7 illustrates various display screens that may be displayed on the display of FIGS. 5 and 6 when using a navigation function of the surgical system;

FIG. 8 illustrates a block diagram of some electrical components of a surgical robot according to some embodiments of the present disclosure;

FIG. 9 illustrates a block diagram of components of a surgical system that includes imaging devices connected to a computer platform which can be operationally connected to a camera tracking system and/or surgical robot according to some embodiments of the present disclosure;

FIG. 10 illustrates an embodiment of a C-Arm imaging device that can be used in combination with the surgical robot in accordance with some embodiments of the present disclosure;

FIG. 11 illustrates an embodiment of an O-Arm imaging device that can be used in combination with the surgical robot in accordance with some embodiments of the present disclosure;

FIG. 12 illustrates a block diagram view of the components of a surgical system that includes a pair of XR headsets in accordance with some embodiments of the present disclosure;

FIG. 13 illustrates an XR headset which is configured with image capture devices in accordance with some embodiments of the present disclosure;

FIG. 14 illustrates an XR headset having additional features in accordance with some embodiments of the present disclosure;

FIG. 15 illustrates electrical components of the XR headset that can be operatively connected to a computer platform, imaging device(s), and/or a surgical robot in accordance with some embodiments of the present disclosure;

FIG. 16 illustrates a block diagram showing arrangement of optical components of the XR headset in accordance with some embodiments of the present disclosure;

FIGS. 17A and 17B illustrate a tether pack connected between the XR headset and a computer platform, in accordance with some embodiments of the present disclosure;

FIGS. 18A and 18B illustrate a collar accessory for the XR headset, in accordance with some embodiments of the present disclosure;

FIG. 19 illustrates components of the collar accessory of FIGS. 18A and 18B, in accordance with some embodiments of the present disclosure;

FIGS. 20 and 20B illustrate a belt accessory for the XR headset, in accordance with some embodiments of the present disclosure; and

FIG. 21 illustrates components of the belt accessory of FIGS. 19A and 19B, in accordance with some embodiments of the present disclosure.

### DETAILED DESCRIPTION

Inventive concepts will now be described more fully hereinafter with reference to the accompanying drawings, in which examples of embodiments of inventive concepts are shown. Inventive concepts may, however, be embodied in many different forms and should not be construed as limited to the embodiments set forth herein. Rather, these embodiments are provided so that this disclosure will be thorough and complete, and will fully convey the scope of various present inventive concepts to those skilled in the art. It should also be noted that these embodiments are not mutually exclusive. Components from one embodiment may be tacitly assumed to be present or used in another embodiment.

Various embodiments disclosed herein are directed to improvements in computer assisted navigation during surgery. An extended reality (XR) headset is operatively connected to the surgical system and configured to provide an interactive environment through which a surgeon, assistant, and/or other personnel can view and select among patient images, view and select among computer generated surgery navigation information, and/or control surgical equipment in the operating room. As will be explained below, the XR headset may be configured to augment a real-world scene with computer generated XR images. The XR headset may be configured to provide an augmented reality (AR) viewing environment by displaying the computer generated XR images on a see-through display screen that allows light from the real-world scene to pass therethrough for combined viewing by the user. Alternatively, the XR headset may be configured to provide a virtual reality (VR) viewing environment by preventing or substantially preventing light from the real-world scene from being directly viewed by the user while the user is viewing the computer generated XR images on a display screen. An XR headset can be configured to provide both AR and VR viewing environments. In one embodiment, both AR and VR viewing environments are provided by lateral bands of substantially differing opacity arranged between the see-through display screen and the real-world scene, so that a VR viewing environment is provided for XR images aligned with a high opacity band and an AR viewing environment is provided for XR images aligned with the low opacity band. In another embodiment, both AR and VR viewing environments are provided by computer adjustable control of an opacity filter that variably constrains how much light from the real-world scene passes through a see-through display screen for combining with the XR images viewed by the user. Thus, the XR headset can also be referred to as an AR headset or a VR headset.

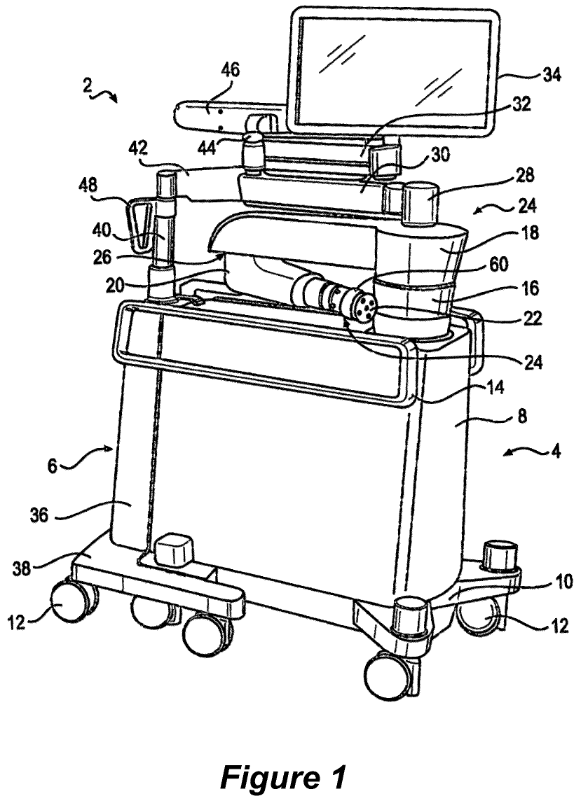

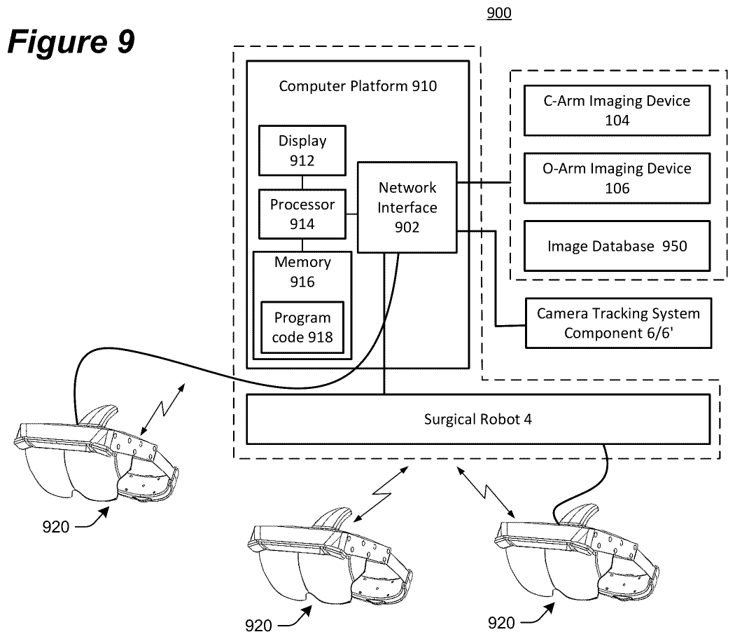

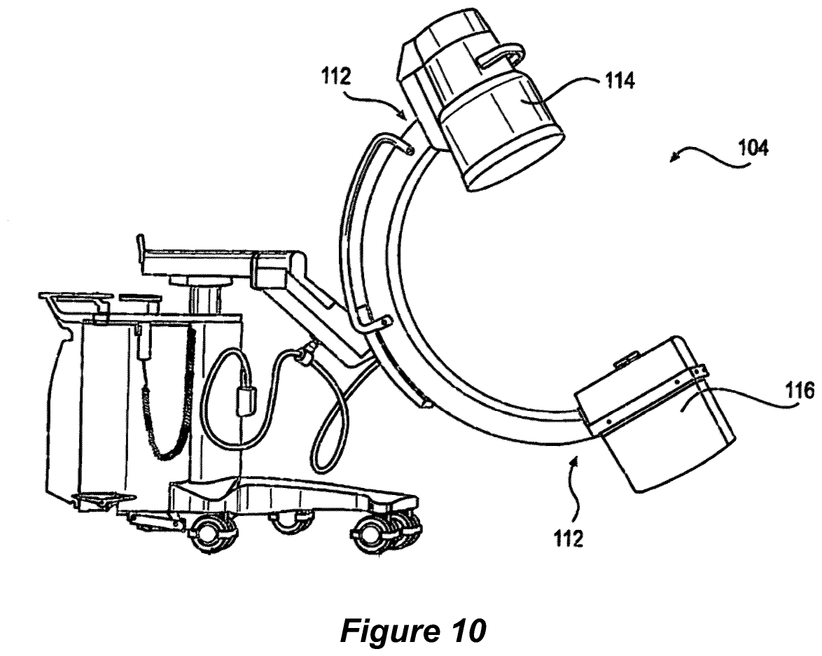

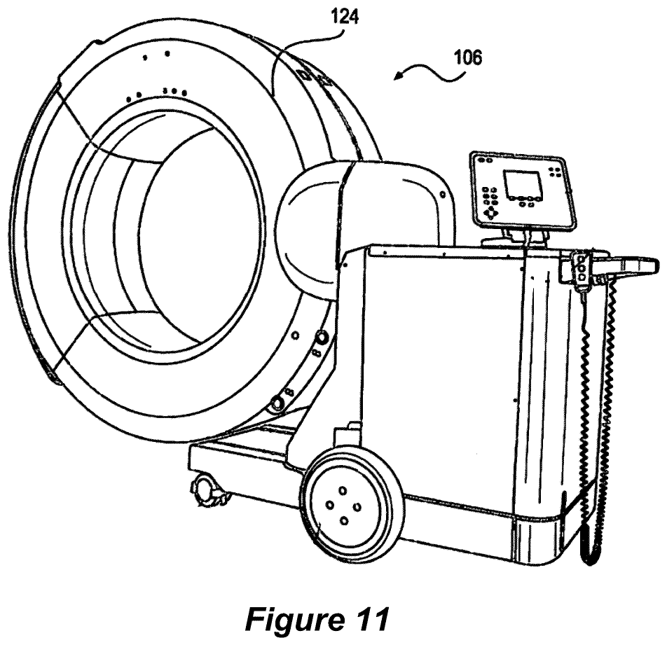

FIG. 1 illustrates an embodiment of a surgical system 2 according to some embodiments of the present disclosure. Prior to performance of an orthopedic or other surgical procedure, a three-dimensional (“3D”) image scan may be taken of a planned surgical area of a patient using, e.g., the C-Arm imaging device 104 of FIG. 10 or O-Arm imaging device 106 of FIG. 11, or from another medical imaging device such as a computed tomography (CT) image or MRI. This scan can be taken pre-operatively (e.g. few weeks before procedure, most common) or intra-operatively. However, any known 3D or 2D image scan may be used in accordance with various embodiments of the surgical system 2. The image scan is sent to a computer platform in communication with the surgical system 2, such as the computer platform 910 of the surgical system 900 (FIG. 9) which may include the camera tracking system component 6, the surgical robot 4 (e.g., robot 2 in FIG. 1), imaging devices (e.g., C-Arm 104, O-Arm 106, etc.), and an image database 950 for storing image scans of patients. A surgeon reviewing the image scan(s) on a display device of the computer platform 910 (FIG. 9) generates a surgical plan defining a target pose for a surgical tool to be used during a surgical procedure on an anatomical structure of the patient. Example surgical tools, also referred to as tools, can include, without limitation, drills, screw drivers, retractors, and implants such as a screws, spacers, interbody fusion devices, plates, rods, etc. In some embodiments, the surgical plan defining the target plane is planned on the 3D image scan displayed on a display device.

As used herein, the term “pose” refers to the position and/or the rotational angle of one object (e.g., dynamic reference array, end effector, surgical tool, anatomical structure, etc.) relative to another object and/or to a defined coordinate system. A pose may therefore be defined based on only the multidimensional position of one object relative to another object and/or to a defined coordinate system, only on the multidimensional rotational angles of the object relative to another object and/or to a defined coordinate system, or on a combination of the multidimensional position and the multidimensional rotational angles. The term “pose” therefore is used to refer to position, rotational angle, or combination thereof.

The surgical system 2 of FIG. 1 can assist surgeons during medical procedures by, for example, holding tools, aligning tools, using tools, guiding tools, and/or positioning tools for use. In some embodiments, surgical system 2 includes a surgical robot 4 and a camera tracking system component 6. The ability to mechanically couple surgical robot 4 and camera tracking system component 6 can allow for surgical system 2 to maneuver and move as a single unit, and allow surgical system 2 to have a small footprint in an area, allow easier movement through narrow passages and around turns, and allow storage within a smaller area.

A surgical procedure may begin with the surgical system 2 moving from medical storage to a medical procedure room. The surgical system 2 may be maneuvered through doorways, halls, and elevators to reach a medical procedure room. Within the room, the surgical system 2 may be physically separated into two separate and distinct systems, the surgical robot 4 and the camera tracking system component 6. Surgical robot 4 may be positioned adjacent the patient at any suitable location to properly assist medical personnel. Camera tracking system component 6 may be positioned at the base of the patient, at the patient shoulders, or any other location suitable to track the present pose and movement of the pose of tracks portions of the surgical robot 4 and the patient. Surgical robot 4 and camera tracking system component 6 may be powered by an onboard power source and/or plugged into an external wall outlet.

Surgical robot 4 may be used to assist a surgeon by holding and/or using tools during a medical procedure. To properly utilize and hold tools, surgical robot 4 may rely on a plurality of motors, computers, and/or actuators to function properly. Illustrated in FIG. 1, robot body 8 may act as the structure in which the plurality of motors, computers, and/or actuators may be secured within surgical robot 4. Robot body 8 may also provide support for robot telescoping support arm 16. The size of robot body 8 may provide a solid platform supporting attached components, and may house, conceal, and protect the plurality of motors, computers, and/or actuators that may operate attached components.

Robot base 10 may act as a lower support for surgical robot 4. In some embodiments, robot base 10 may support robot body 8 and may attach robot body 8 to a plurality of powered wheels 12. This attachment to wheels may allow robot body 8 to move in space efficiently. Robot base 10 may run the length and width of robot body 8. Robot base 10 may be about two inches to about 10 inches tall. Robot base 10 may cover, protect, and support powered wheels 12.

In some embodiments, as illustrated in FIG. 1, at least one powered wheel 12 may be attached to robot base 10. Powered wheels 12 may attach to robot base 10 at any location. Each individual powered wheel 12 may rotate about a vertical axis in any direction. A motor may be disposed above, within, or adjacent to powered wheel 12. This motor may allow for surgical system 2 to maneuver into any location and stabilize and/or level surgical system 2. A rod, located within or adjacent to powered wheel 12, may be pressed into a surface by the motor. The rod, not pictured, may be made of any suitable metal to lift surgical system 2. The rod may lift powered wheel 12, which may lift surgical system 2, to any height required to level or otherwise fix the orientation of the surgical system 2 in relation to a patient. The weight of surgical system 2, supported through small contact areas by the rod on each wheel, prevents surgical system 2 from moving during a medical procedure. This rigid positioning may prevent objects and/or people from moving surgical system 2 by accident.

Moving surgical system 2 may be facilitated using robot railing 14. Robot railing 14 provides a person with the ability to move surgical system 2 without grasping robot body 8. As illustrated in FIG. 1, robot railing 14 may run the length of robot body 8, shorter than robot body 8, and/or may run longer the length of robot body 8. Robot railing 14 may further provide protection to robot body 8, preventing objects and or personnel from touching, hitting, or bumping into robot body 8.

Robot body 8 may provide support for a Selective Compliance Articulated Robot Arm, hereafter referred to as a “SCARA.” A SCARA 24 may be beneficial to use within the surgical system 2 due to the repeatability and compactness of the robotic arm. The compactness of a SCARA may provide additional space within a medical procedure, which may allow medical professionals to perform medical procedures free of excess clutter and confining areas. SCARA 24 may comprise robot telescoping support 16, robot support arm 18, and/or robot arm 20. Robot telescoping support 16 may be disposed along robot body 8. As illustrated in FIG. 1, robot telescoping support 16 may provide support for the SCARA 24 and display 34. In some embodiments, robot telescoping support 16 may extend and contract in a vertical direction. The body of robot telescoping support 16 may be any width and/or height configured to support the stress and weight placed upon it.

In some embodiments, medical personnel may move SCARA 24 through a command submitted by the medical personnel. The command may originate from input received on display 34, a tablet, and/or an XR headset (e.g., headset 920 in FIG. 9) as will be explained in further detail below. The XR headset may eliminate the need for medical personnel to refer to any other display such as the display 34 or a tablet, which enables the SCARA 24 to be configured without the display 34 and/or the tablet. The command may be generated by the depression of a switch and/or the depression of a plurality of switches, and/or may be generated based on a hand gesture command and/or voice command that is sensed by the XR headset as will be explained in further detail below.

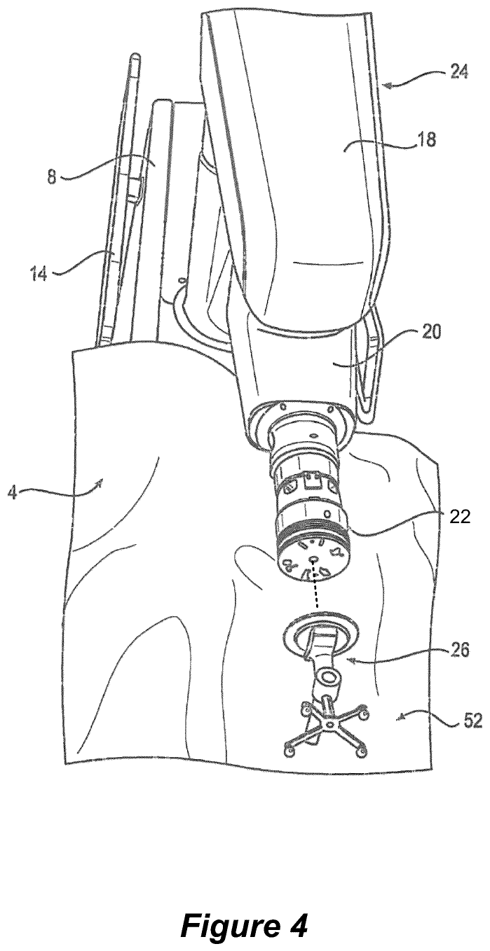

As shown in FIG. 5, an activation assembly 60 may include a switch and/or a plurality of switches. The activation assembly 60 may be operable to transmit a move command to the SCARA 24 allowing an operator to manually manipulate the SCARA 24. When the switch, or plurality of switches, is depressed the medical personnel may have the ability to move SCARA 24 through applied hand movements. Alternatively or additionally, an operator may control movement of the SCARA 24 through hand gesture commands and/or voice commands that are sensed by the XR headset as will be explained in further detail below. Additionally, when the SCARA 24 is not receiving a command to move, the SCARA 24 may lock in place to prevent accidental movement by personnel and/or other objects. By locking in place, the SCARA 24 provides a solid platform through which the end effector 26 can guide a surgical tool during a medical procedure.

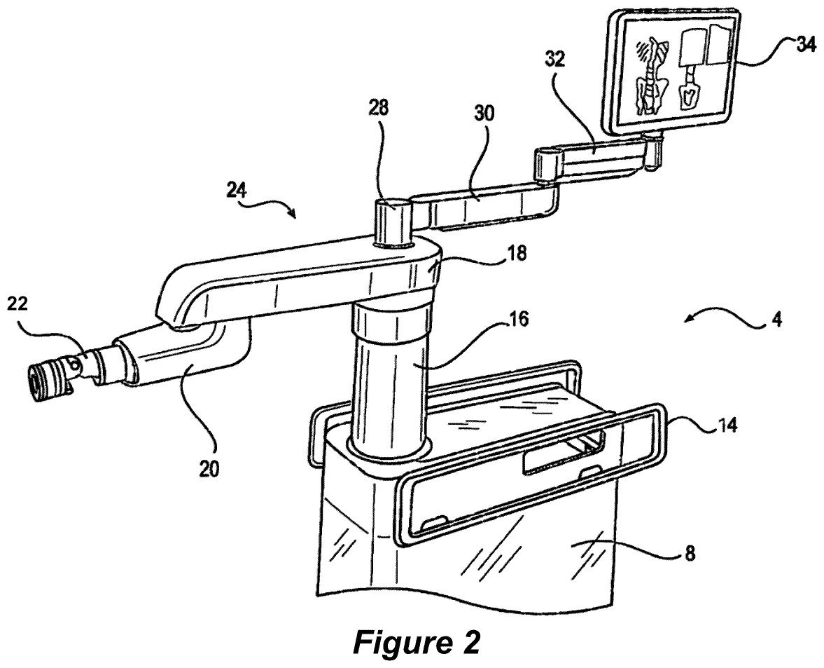

Robot support arm 18 can be connected to robot telescoping support 16 by various mechanisms. In some embodiments, best seen in FIGS. 1 and 2, robot support arm 18 rotates in any direction in regard to robot telescoping support 16. Robot support arm 18 may rotate three hundred and sixty degrees around robot telescoping support 16. Robot arm 20 may connect to robot support arm 18 at any suitable location and by various mechanisms that enable rotation in any direction relative to robot support arm 18. In one embodiment, the robot arm 20 can rotate three hundred and sixty degrees relative to the robot support arm 18. This free rotation allows an operator to position robot arm 20 according to a surgical plan.

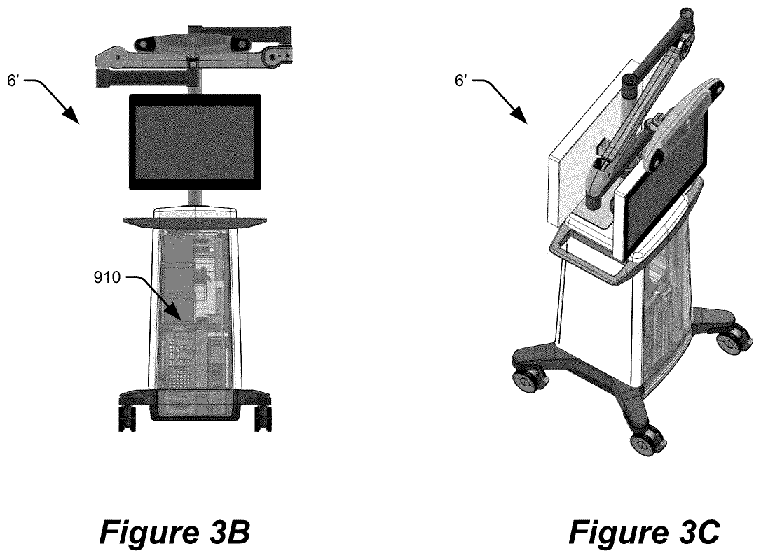

The end effector 26 shown in FIGS. 4 and 5 may attach to robot arm 20 in any suitable location. The end effector 26 can be configured to attach to an end effector coupler 22 of the robot arm 20 positioned by the surgical robot 4. The example end effector 26 includes a tubular guide that guides movement of an inserted surgical tool relative to an anatomical structure on which a surgical procedure is to be performed.

In some embodiments, a dynamic reference array 52 is attached to the end effector 26. Dynamic reference arrays, also referred to as “DRAs” herein, are rigid bodies which may be disposed on an anatomical structure (e.g., bone) of a patient, one or more XR headsets being worn by personnel in the operating room, the end effector, the surgical robot, a surgical tool in a navigated surgical procedure. The computer platform 910 in combination with the camera tracking system component 6 or other 3D localization system are configured to track in real-time the pose (e.g., positions and rotational orientations) of the DRA. The DRA can include fiducials, such as the illustrated arrangement of balls. This tracking of 3D coordinates of the DRA can allow the surgical system 2 to determine the pose of the DRA in any multidimensional space in relation to the target anatomical structure of the patient 50 in FIG. 5.

As illustrated in FIG. 1, a light indicator 28 may be positioned on top of the SCARA 24. Light indicator 28 may illuminate as any type of light to indicate “conditions” in which surgical system 2 is currently operating. In some embodiments, the light may be produced by LED bulbs, which may form a ring around light indicator 28. Light indicator 28 may comprise a fully permeable material that can let light shine through the entirety of light indicator 28. Light indicator 28 may be attached to lower display support 30. Lower display support 30, as illustrated in FIG. 2 may allow an operator to maneuver display 34 to any suitable location. Lower display support 30 may attach to light indicator 28 by any suitable mechanism. In some embodiments, lower display support 30 may rotate about light indicator 28 or be rigidly attached thereto. Upper display support 32 may attach to lower display support 30 by any suitable mechanism.

In some embodiments, a tablet may be used in conjunction with display 34 and/or without display 34. The tablet may be disposed on upper display support 32, in place of display 34, and may be removable from upper display support 32 during a medical operation. In addition the tablet may communicate with display 34. The tablet may be able to connect to surgical robot 4 by any suitable wireless and/or wired connection. In some embodiments, the tablet may be able to program and/or control surgical system 2 during a medical operation. When controlling surgical system 2 with the tablet, all input and output commands may be duplicated on display 34. The use of a tablet may allow an operator to manipulate surgical robot 4 without having to move around patient 50 and/or to surgical robot 4.

As will be explained below, in some embodiments a surgeon and/or other personnel can wear XR headsets that may be used in conjunction with display 34 and/or a tablet or the XR head(s) may eliminate the need for use of the display 34 and/or tablet.

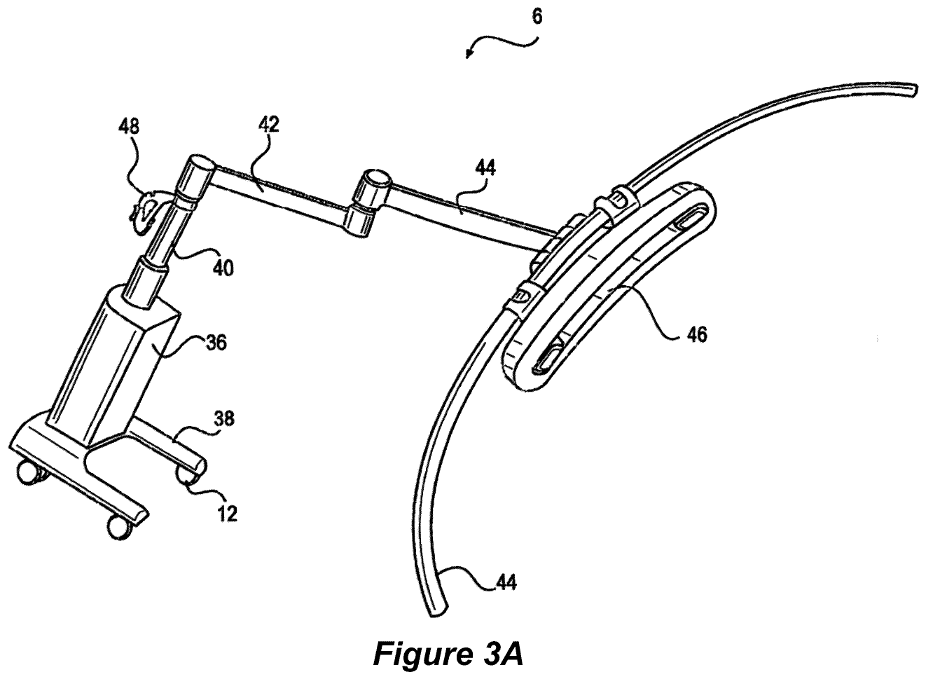

As illustrated in FIGS. 3A and 5, camera tracking system component 6 works in conjunction with surgical robot 4 through wired or wireless communication networks. Referring to FIGS. 1, 3 and 5, camera tracking system component 6 can include some similar components to the surgical robot 4. For example, camera body 36 may provide the functionality found in robot body 8. Robot body 8 may provide an auxiliary tracking bar upon which cameras 46 are mounted. The structure within robot body 8 may also provide support for the electronics, communication devices, and power supplies used to operate camera tracking system component 6. Camera body 36 may be made of the same material as robot body 8. Camera tracking system component 6 may communicate directly to an XR headset, tablet and/or display 34 by a wireless and/or wired network to enable the XR headset, tablet and/or display 34 to control the functions of camera tracking system component 6.

Camera body 36 is supported by camera base 38. Camera base 38 may function as robot base 10. In the embodiment of FIG. 1, camera base 38 may be wider than robot base 10. The width of camera base 38 may allow for camera tracking system component 6 to connect with surgical robot 4. As illustrated in FIG. 1, the width of camera base 38 may be large enough to fit outside robot base 10. When camera tracking system component 6 and surgical robot 4 are connected, the additional width of camera base 38 may allow surgical system 2 additional maneuverability and support for surgical system 2.

As with robot base 10, a plurality of powered wheels 12 may attach to camera base 38. Powered wheel 12 may allow camera tracking system component 6 to stabilize and level or set fixed orientation in regards to patient 50, similar to the operation of robot base 10 and powered wheels 12. This stabilization may prevent camera tracking system component 6 from moving during a medical procedure and may keep cameras 46 on the auxiliary tracking bar from losing track of a DRA connected to an XR headset and/or the surgical robot 4, and/or losing track of one or more DRAs 52 connected to an anatomical structure 54 and/or tool 58 within a designated area 56 as shown in FIGS. 3A and 5. This stability and maintenance of tracking enhances the ability of surgical robot 4 to operate effectively with camera tracking system component 6. Additionally, the wide camera base 38 may provide additional support to camera tracking system component 6. Specifically, a wide camera base 38 may prevent camera tracking system component 6 from tipping over when cameras 46 is disposed over a patient, as illustrated in FIGS. 3A and 5.

Camera telescoping support 40 may support cameras 46 on the auxiliary tracking bar. In some embodiments, telescoping support 40 moves cameras 46 higher or lower in the vertical direction. Camera handle 48 may be attached to camera telescoping support 40 at any suitable location and configured to allow an operator to move camera tracking system component 6 into a planned position before a medical operation. In some embodiments, camera handle 48 is used to lower and raise camera telescoping support 40. Camera handle 48 may perform the raising and lowering of camera telescoping support 40 through the depression of a button, switch, lever, and/or any combination thereof.

Lower camera support arm 42 may attach to camera telescoping support 40 at any suitable location, in embodiments, as illustrated in FIG. 1, lower camera support arm 42 may rotate three hundred and sixty degrees around telescoping support 40. This free rotation may allow an operator to position cameras 46 in any suitable location. Lower camera support arm 42 may connect to telescoping support 40 by any suitable mechanism. Lower camera support arm 42 may be used to provide support for cameras 46. Cameras 46 may be attached to lower camera support arm 42 by any suitable mechanism. Cameras 46 may pivot in any direction at the attachment area between cameras 46 and lower camera support arm 42. In embodiments a curved rail 44 may be disposed on lower camera support arm 42.

Curved rail 44 may be disposed at any suitable location on lower camera support arm 42. As illustrated in FIG. 3A, curved rail 44 may attach to lower camera support arm 42 by any suitable mechanism. Curved rail 44 may be of any suitable shape, a suitable shape may be a crescent, circular, oval, elliptical, and/or any combination thereof. Cameras 46 may be moveably disposed along curved rail 44. Cameras 46 may attach to curved rail 44 by, for example, rollers, brackets, braces, motors, and/or any combination thereof. Motors and rollers, not illustrated, may be used to move cameras 46 along curved rail 44. As illustrated in FIG. 3A, during a medical procedure, if an object prevents cameras 46 from viewing one or more DRAs being tracked, the motors may responsively move cameras 46 along curved rail 44. This motorized movement may allow cameras 46 to move to a new position that is no longer obstructed by the object without moving camera tracking system component 6. While cameras 46 is obstructed from viewing one or more tracked DRAs, camera tracking system component 6 may send a stop signal to a surgical robot 4, XR headset, display 34, and/or a tablet. The stop signal may prevent SCARA 24 from moving until cameras 46 has reacquired tracked DRAs 52 and/or can warn an operator wearing the XR headset and/or viewing the display 34 and/or the tablet. This SCARA 24 can be configured to respond to receipt of a stop signal by stopping further movement of the base and/or end effector coupler 22 until the camera tracking system can resume tracking of DRAs.

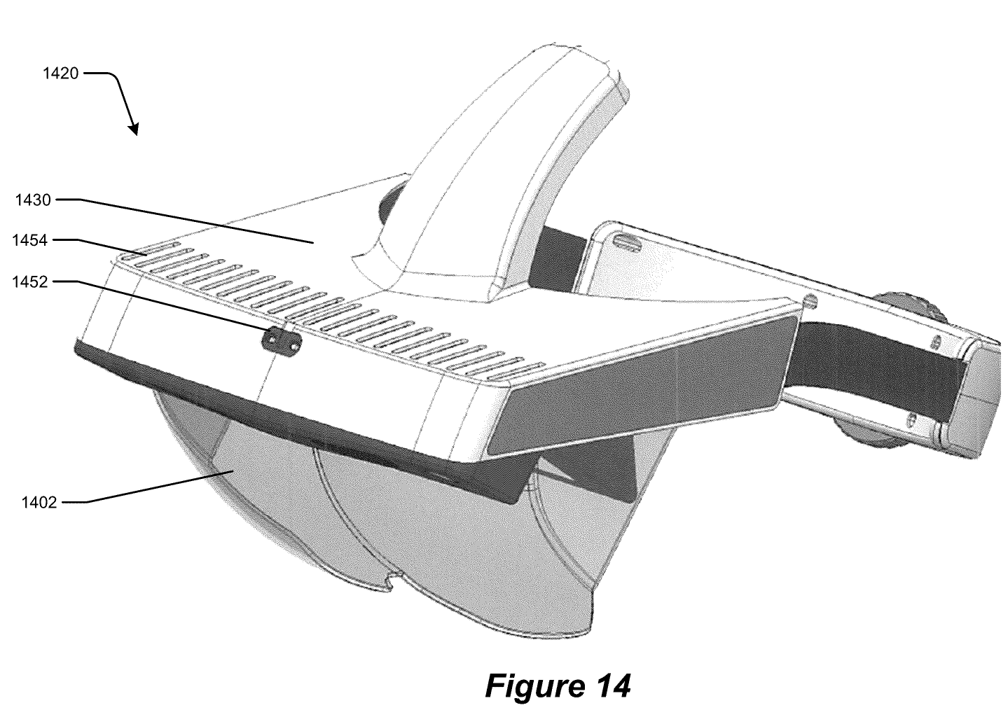

FIGS. 3B and 3C illustrate a front view and isometric view of another camera tracking system component 6′ which may be used with the surgical system of FIG. 1 or may be used independent of a surgical robot. For example, the camera tracking system component 6′ may be used for providing navigated surgery without use of robotic guidance. One of the differences between the camera tracking system component 6′ of FIGS. 3B and 3C and the camera tracking system component 6 of FIG. 3A, is that the camera tracking system component 6′ of FIGS. 3B and 3C includes a housing that transports the computer platform 910. The computer platform 910 can be configured to perform camera tracking operations to track DRAs, perform navigated surgery operations that provide surgical navigation information to a display device, e.g., XR headset and/or other display device, and perform other computational operations disclosed herein. The computer platform 910 can therefore include a navigation computer, such as one or more of the navigation computers of FIGS. 14.

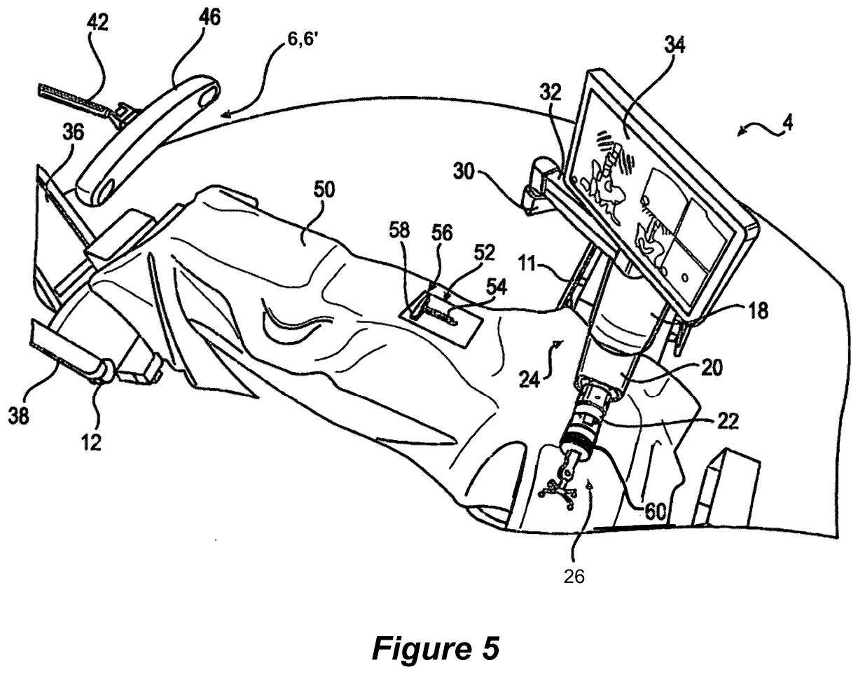

FIG. 6 illustrates a block diagram view of the components of the surgical system of FIG. 5 used for the medical operation. Referring to FIG. 6, the navigation cameras 46 on the auxiliary tracking bar has a navigation field-of-view 600 in which the pose (e.g., position and orientation) of the reference array 602 attached to the patient, the reference array 604 attached to the surgical instrument, and the robot arm 20 are tracked. The navigation cameras 46 may be part of the camera tracking system component 6′ of FIGS. 3B and 3C, which includes the computer platform 910 configured to perform the operations described below. The reference arrays enable tracking by reflecting light in known patterns, which are decoded to determine their respective poses by the tracking subsystem of the surgical robot 4. If the line-of-sight between the patient reference array 602 and the navigation cameras 46 in the auxiliary tracking bar is blocked (for example, by a medical personnel, instrument, etc.), further navigation of the surgical instrument may not be able to be performed and a responsive notification may temporarily halt further movement of the robot arm 20 and surgical robot 4, display a warning on the display 34, and/or provide an audible warning to medical personnel. The display 34 is accessible to the surgeon 610 and assistant 612 but viewing requires a head to be turned away from the patient and for eye focus to be changed to a different distance and location. The navigation software may be controlled by a tech personnel 614 based on vocal instructions from the surgeon.

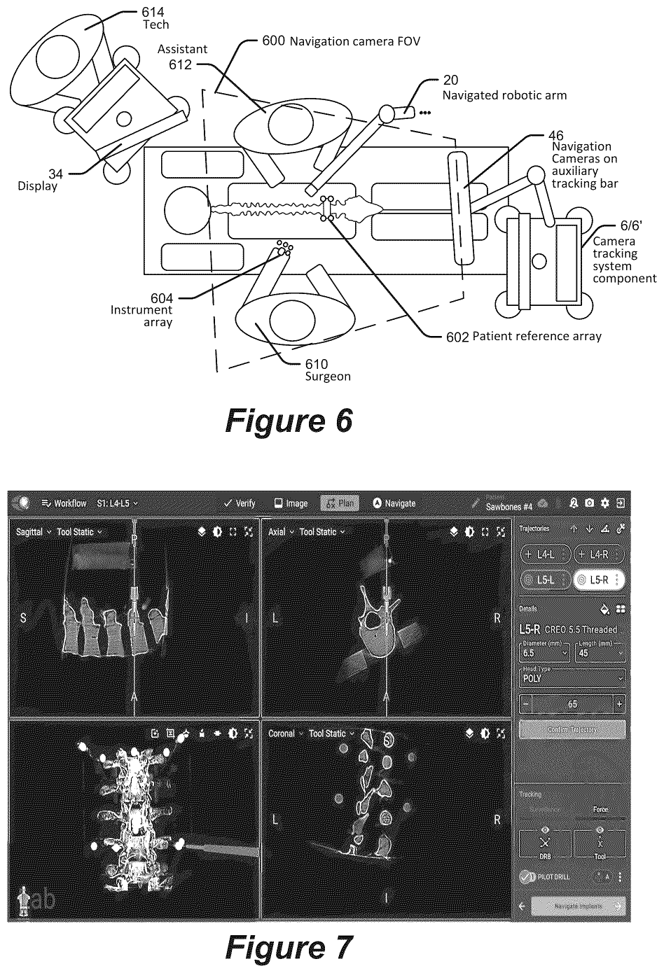

FIG. 7 illustrates various display screens that may be displayed on the display 34 of FIGS. 5 and 6 by the surgical robot 4 when using a navigation function of the surgical system 2. The display screens can include, without limitation, patient radiographs with overlaid graphical representations of models of instruments that are positioned in the display screens relative to the anatomical structure based on a developed surgical plan and/or based on poses of tracked reference arrays, various user selectable menus for controlling different stages of the surgical procedure and dimension parameters of a virtually projected implant (e.g. length, width, and/or diameter).

For navigated surgery, various processing components (e.g., computer platform 910) and associated software described below are provided that enable pre-operatively planning of a surgical procedure, e.g., implant placement, and electronic transfer of the plan to computer platform 910 to provide navigation information to one or more users during the planned surgical procedure.

For robotic navigation, various processing components (e.g., computer platform 910) and associated software described below are provided that enable pre-operatively planning of a surgical procedure, e.g., implant placement, and electronic transfer of the plan to the surgical robot 4. The surgical robot 4 uses the plan to guide the robot arm 20 and connected end effector 26 to provide a target pose for a surgical tool relative to a patient anatomical structure for a step of the planned surgical procedure.

Various embodiments below are directed to using one or more XR headsets that can be worn by the surgeon 610, the assistant 612, and/or other medical personnel to provide an improved user interface for receiving information from and/or providing control commands to the surgical robot, the camera tracking system component 6/6′, and/or other medical equipment in the operating room.

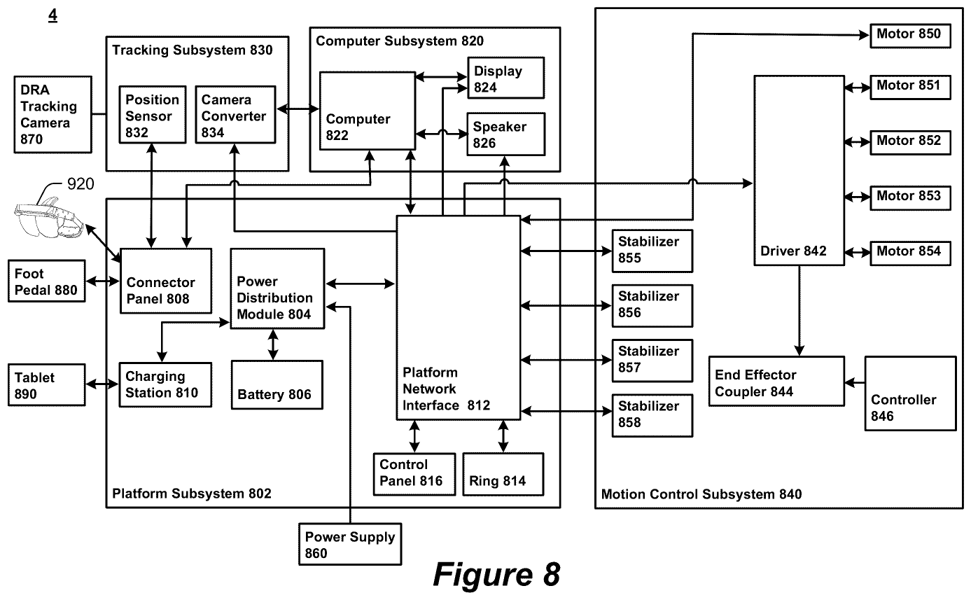

FIG. 8 illustrates a block diagram of some electrical components of the surgical robot 4 according to some embodiments of the present disclosure. Referring to FIG. 8, a load cell (not shown) may be configured to track force applied to end effector coupler 22. In some embodiments the load cell may communicate with a plurality of motors 850, 851, 852, 853, and/or 854. As load cell senses force, information as to the amount of force applied may be distributed from a switch array and/or a plurality of switch arrays to a controller 846. Controller 846 may take the force information from load cell and process it with a switch algorithm. The switch algorithm is used by the controller 846 to control a motor driver 842. The motor driver 842 controls operation of one or more of the motors 850, 851, 852, 853, and 854. Motor driver 842 may direct a specific motor to produce, for example, an equal amount of force measured by load cell through the motor. In some embodiments, the force produced may come from a plurality of motors, e.g., 850-854, as directed by controller 846. Additionally, motor driver 842 may receive input from controller 846. Controller 846 may receive information from load cell as to the direction of force sensed by load cell. Controller 846 may process this information using a motion controller algorithm. The algorithm may be used to provide information to specific motor drivers 842. To replicate the direction of force, controller 846 may activate and/or deactivate certain motor drivers 842. Controller 846 may control one or more motors, e.g. one or more of 850-854, to induce motion of end effector 26 in the direction of force sensed by load cell. This force-controlled motion may allow an operator to move SCARA 24 and end effector 26 effortlessly and/or with very little resistance. Movement of end effector 26 can be performed to position end effector 26 in any suitable pose (i.e., location and angular orientation relative to defined three-dimensional (3D) orthogonal reference axes) for use by medical personnel.

Activation assembly 60, best illustrated in FIG. 5, may form of a bracelet that wraps around end effector coupler 22. The activation assembly 60 may be located on any part of SCARA 24, any part of end effector coupler 22, may be worn by medical personnel (and communicate wirelessly), and/or any combination thereof. Activation assembly 60 may comprise of a primary button and a secondary button.

Depressing primary button may allow an operator to move SCARA 24 and end effector coupler 22. According to one embodiment, once set in place, SCARA 24 and end effector coupler 22 may not move until an operator programs surgical robot 4 to move SCARA 24 and end effector coupler 22, or is moved using primary button. In some examples, it may require the depression of at least two non-adjacent primary activation switches before SCARA 24 and end effector coupler 22 will respond to operator commands. Depression of at least two primary activation switches may prevent the accidental movement of SCARA 24 and end effector coupler 22 during a medical procedure.

Activated by primary button, load cell may measure the force magnitude and/or direction exerted upon end effector coupler 22 by an operator, i.e. medical personnel. This information may be transferred to one or more motors, e.g. one or more of 850-854, within SCARA 24 that may be used to move SCARA 24 and end effector coupler 22. Information as to the magnitude and direction of force measured by load cell may cause the one or more motors, e.g. one or more of 850-854, to move SCARA 24 and end effector coupler 22 in the same direction as sensed by the load cell. This force-controlled movement may allow the operator to move SCARA 24 and end effector coupler 22 easily and without large amounts of exertion due to the motors moving SCARA 24 and end effector coupler 22 at the same time the operator is moving SCARA 24 and end effector coupler 22.

In some examples, a secondary button may be used by an operator as a “selection” device. During a medical operation, surgical robot 4 may notify medical personnel to certain conditions by the XR headset(s) 920, display 34 and/or light indicator 28. The XR headset(s) 920 are each configured to display images on a see-through display screen to form an extended reality image that is overlaid on real-world objects viewable through the see-through display screen. Medical personnel may be prompted by surgical robot 4 to select a function, mode, and/or asses the condition of surgical system 2. Depressing secondary button a single time may activate certain functions, modes, and/or acknowledge information communicated to medical personnel through the XR headset(s) 920, display 34 and/or light indicator 28. Additionally, depressing the secondary button multiple times in rapid succession may activate additional functions, modes, and/or select information communicated to medical personnel through the XR headset(s) 920, display 34 and/or light indicator 28.

With further reference to FIG. 8, electrical components of the surgical robot 4 include platform subsystem 802, computer subsystem 820, motion control subsystem 840, and tracking subsystem 830. Platform subsystem 802 includes battery 806, power distribution module 804, connector panel 808, and charging station 810. Computer subsystem 820 includes computer 822, display 824, and speaker 826. Motion control subsystem 840 includes driver circuit 842, motors 850, 851, 852, 853, 854, stabilizers 855, 856, 857, 858, end effector connector 844, and controller 846. Tracking subsystem 830 includes position sensor 832 and camera converter 834. Surgical robot 4 may also include a removable foot pedal 880 and removable tablet computer 890.

Input power is supplied to surgical robot 4 via a power source which may be provided to power distribution module 804. Power distribution module 804 receives input power and is configured to generate different power supply voltages that are provided to other modules, components, and subsystems of surgical robot 4. Power distribution module 804 may be configured to provide different voltage supplies to connector panel 808, which may be provided to other components such as computer 822, display 824, speaker 826, driver 842 to, for example, power motors 850-854 and end effector connector 844, and provided to camera converter 834 and other components for surgical robot 4. Power distribution module 804 may also be connected to battery 806, which serves as temporary power source in the event that power distribution module 804 does not receive power from an input power. At other times, power distribution module 804 may serve to charge battery 806.

Connector panel 808 may serve to connect different devices and components to surgical robot 4 and/or associated components and modules. Connector panel 808 may contain one or more ports that receive lines or connections from different components. For example, connector panel 808 may have a ground terminal port that may ground surgical robot 4 to other equipment, a port to connect foot pedal 880, a port to connect to tracking subsystem 830, which may include position sensor 832, camera converter 834, and DRA tracking cameras 870. Connector panel 808 may also include other ports to allow USB, Ethernet, HDMI communications to other components, such as computer 822. In accordance with some embodiments, the connector panel 808 can include a wired and/or wireless interface for operatively connecting one or more XR headsets 920 to the tracking subsystem 830 and/or the computer subsystem 820.

Control panel 816 may provide various buttons or indicators that control operation of surgical robot 4 and/or provide information from surgical robot 4 for observation by an operator. For example, control panel 816 may include buttons to power on or off surgical robot 4, lift or lower support 16, and lift or lower stabilizers 855-858 that may be designed to engage powered wheels 12 (e.g., casters) to lock surgical robot 4 from physically moving. Other buttons may stop surgical robot 4 in the event of an emergency, which may remove all motor power and apply mechanical brakes to stop all motion from occurring. Control panel 816 may also have indicators notifying the operator of certain system conditions such as a line power indicator or status of charge for battery 806. In accordance with some embodiments, one or more XR headsets 920 may communicate, e.g. via the connector panel 808, to control operation of the surgical robot 4 and/or to received and display information generated by surgical robot 4 for observation by persons wearing the XR headsets 920.

Computer 822 of computer subsystem 820 includes an operating system and software to operate assigned functions of surgical robot 4. Computer 822 may receive and process information from other components (for example, tracking subsystem 830, platform subsystem 802, and/or motion control subsystem 840) in order to display information to the operator. Further, computer subsystem 820 may provide output through the speaker 826 for the operator. The speaker may be part of the surgical robot, part of an XR headset 920, or within another component of the surgical system 2. The display 824 may correspond to the display 34 shown in FIGS. 1 and 2.

Tracking subsystem 830 may include position sensor 832 and camera converter 834. Tracking subsystem 830 may correspond to the camera tracking system component 6 of FIG. 3. The DRA tracking cameras 870 operate with the position sensor 832 to determine the pose of DRAs 52. This tracking may be conducted in a manner consistent with the present disclosure including the use of infrared or visible light technology that tracks the location of active or passive elements of DRAs 52, such as LEDs or reflective markers, respectively.

Functional operations of the tracking subsystem 830 and the computer subsystem 820 can be included in the computer platform 910, which can be transported by the camera tracking system component 6′ of FIGS. 3A and 3B. The tracking subsystem 830 can be configured to determine the poses, e.g., location and angular orientation of the tracked DRAs. The computer platform 910 can also include a navigation controller that is configured to use the determined poses to provide navigation information to users that guides their movement of tracked tools relative to position-registered patient images and/or tracked anatomical structures during a planned surgical procedure. The computer platform 910 can display information on the display of FIGS. 3B and 3C and/or to one or more XR headsets 920. The computer platform 910, when used with a surgical robot, can be configured to communicate with the computer subsystem 820 and other subsystems of FIG. 8 to control movement of the end effector 26. For example, as will be explained below the computer platform 910 can generate a graphical representation of a patient's anatomical structure, surgical tool, user's hand, etc. with a displayed size, shape, color, and/or pose that is controlled based on the determined pose(s) of one or more the tracked DRAs, and which the graphical representation that is displayed can be dynamically modified to track changes in the determined poses over time.

Motion control subsystem 840 may be configured to physically move support 16 (e.g., vertical column), upper arm 18, lower arm 20, or rotate end effector coupler 22. The physical movement may be conducted through the use of one or more motors 850-854. For example, motor 850 may be configured to vertically lift or lower support 16. Motor 851 may be configured to laterally move upper arm 18 around a point of engagement with vertical column 16 as shown in FIG. 2. Motor 852 may be configured to laterally move lower arm 20 around a point of engagement with upper arm 18 as shown in FIG. 2. Motors 853 and 854 may be configured to move end effector coupler 22 to provide translational movement and rotation along in about three-dimensional axes. The computer platform 910 shown in FIG. 9 can provide control input to the controller 846 that guides movement of the end effector coupler 22 to position a passive end effector, which is connected thereto, with a planned pose (i.e., location and angular orientation relative to defined 3D orthogonal reference axes) relative to an anatomical structure that is to be operated on during a planned surgical procedure. Motion control subsystem 840 may be configured to measure position of the end effector coupler 22 and/or the end effector 26 using integrated position sensors (e.g. encoders).

FIG. 9 illustrates a block diagram of components of a surgical system that includes imaging devices (e.g., C-Arm 104, O-Arm 106, etc.) connected to a computer platform 910 which can be operationally connected to a camera tracking system component 6 (FIG. 3A) or 6′ (FIGS. 3B, 3C) and/or to surgical robot 4 according to some embodiments of the present disclosure. Alternatively, at least some operations disclosed herein as being performed by the computer platform 910 may additionally or alternatively be performed by components of a surgical system.

Referring to FIG. 9, the computer platform 910 includes a display 912, at least one processor circuit 914 (also referred to as a processor for brevity), at least one memory circuit 916 (also referred to as a memory for brevity) containing computer readable program code 918, and at least one network interface 902 (also referred to as a network interface for brevity). The display 912 may be part of an XR headset 920 in accordance with some embodiments of the present disclosure. The network interface 902 can be configured to connect to a C-Arm imaging device 104 in FIG. 10, an O-Arm imaging device 106 in FIG. 11, another medical imaging device, an image database 950 containing patient medical images, components of the surgical robot 4, and/or other electronic equipment.

When used with a surgical robot 4, the display 912 may correspond to the display 34 of FIG. 2 and/or the tablet 890 of FIG. 8 and/or the XR headset 920 that is operatively connected to the surgical robot 4, the network interface 902 may correspond to the platform network interface 812 of FIG. 8, and the processor 914 may correspond to the computer 822 of FIG. 8. The network interface 902 of the XR headset 920 may be configured to communicate through a wired network, e.g., thin wire ethernet, and/or through wireless RF transceiver link according to one or more wireless communication protocols, e.g., WLAN, 3GPP 4G and/or 5G (New Radio) cellular communication standards, etc.

The processor 914 may include one or more data processing circuits, such as a general purpose and/or special purpose processor, e.g., microprocessor and/or digital signal processor. The processor 914 is configured to execute the computer readable program code 918 in the memory 916 to perform operations, which may include some or all of the operations described herein as being performed for surgery planning, navigated surgery, and/or robotic surgery.

The computer platform 910 can be configured to provide surgery planning functionality. The processor 914 can operate to display on the display 912 and/or on the XR headset 920 an image of an anatomical structure, e.g., vertebra, that is received from one of the imaging devices 104 and 106 and/or from the image database 950 through the network interface 902. The processor 914 receives an operator's definition of where the anatomical structure shown in one or more images is to have a surgical procedure, e.g., screw placement, such as by the operator touch selecting locations on the display 912 for planned procedures or using a mouse-based cursor to define locations for planned procedures. When the image is displayed in the XR headset 920, the XR headset can be configured to sense in gesture-based commands formed by the wearer and/or sense voice based commands spoken by the wearer, which can be used to control selection among menu items and/or control how objects are displayed on the XR headset 920 as will be explained in further detail below.

The computer platform 910 can be configured to enable anatomy measurement, which can be particularly useful for knee surgery, like measurement of various angles determining center of hip, center of angles, natural landmarks (e.g. transepicondylar line, Whitesides line, posterior condylar line), etc. Some measurements can be automatic while some others can involve human input or assistance. The computer platform 910 may be configured to allow an operator to input a choice of the correct implant for a patient, including choice of size and alignment. The computer platform 910 may be configured to perform automatic or semi-automatic (involving human input) segmentation (image processing) for CT images or other medical images. The surgical plan for a patient may be stored in a cloud-based server, which may correspond to database 950, for retrieval by the surgical robot 4.

During orthopedic surgery, for example, a surgeon may choose which cut to make (e.g. posterior femur, proximal tibia etc.) using a computer screen (e.g. touchscreen) or extended reality (XR) interaction (e.g., hand gesture based commands and/or voice based commands) via, e.g., the XR headset 920. The computer platform 910 can generate navigation information which provides visual guidance to the surgeon for performing the surgical procedure. When used with the surgical robot 4, the computer platform 910 can provide guidance that allows the surgical robot 4 to automatically move the end effector 26 to a target pose so that the surgical tool is aligned with a target location to perform the surgical procedure on an anatomical structure.

In some embodiments, the surgical system 900 can use two DRAs to track patient anatomy position, such as one connected to patient tibia and one connected to patient femur. The system 900 may use standard navigated instruments for the registration and checks (e.g. a pointer similar to the one used in Globus ExcelsiusGPS™ system for spine surgery).

A particularly challenging task in navigated surgery is how to plan the position of an implant in spine, knee, and other anatomical structures where surgeons struggle to perform the task on a computer screen which is a 2D representation of the 3D anatomical structure. The system 900 could address this problem by using the XR headset 920 to display a three-dimensional (3D) computer generated representations of the anatomical structure and a candidate implant device. The computer generated representations are scaled and posed relative to each other on the display screen under guidance of the computer platform 910 and which can be manipulated by a surgeon while viewed through the XR headset 920. A surgeon may, for example, manipulate the displayed computer-generated representations of the anatomical structure, the implant, a surgical tool, etc., using hand gesture based commands and/or voice based commands that are sensed by the XR headset 920.

For example, a surgeon can view a displayed virtual handle on a virtual implant, and can manipulate (e.g., grab and move) the virtual handle to move the virtual implant to a desired pose and adjust a planned implant placement relative to a graphical representation of an anatomical structure. Afterward, during surgery, the computer platform 910 could display navigation information through the XR headset 920 that facilitates the surgeon's ability to more accurately follow the surgical plan to insert the implant and/or to perform another surgical procedure on the anatomical structure. When the surgical procedure involves bone removal, the progress of bone removal, e.g., depth of cut, can be displayed in real-time through the XR headset 920. Other features that may be displayed through the XR headset 920 can include, without limitation, gap or ligament balance along a range of joint motion, contact line on the implant along the range of joint motion, ligament tension and/or laxity through color or other graphical renderings, etc.

The computer platform 910, in some embodiments, can allow planning for use of standard surgical tools and/or implants, e.g., posterior stabilized implants and cruciate retaining implants, cemented and cementless implants, revision systems for surgeries related to, for example, total or partial knee and/or hip replacement and/or trauma.

An automated imaging system can be used in conjunction with the computer platform 910 to acquire pre-operative, intra-operative, post-operative, and/or real-time image data of an anatomical structure. Example automated imaging systems are illustrated in FIGS. 10 and 11. In some embodiments, the automated imaging system is a C-arm 104 (FIG. 10) imaging device or an O-arm® 106 (FIG. 11). (O-arm® is copyrighted by Medtronic Navigation, Inc. having a place of business in Louisville, Colo., USA). It may be desirable to take x-rays of a patient from a number of different positions, without the need for frequent manual repositioning of the patient which may be required in an x-ray system. C-arm 104 x-ray diagnostic equipment may solve the problems of frequent manual repositioning and may be well known in the medical art of surgical and other interventional procedures. As illustrated in FIG. 10, a C-arm includes an elongated C-shaped member terminating in opposing distal ends 112 of the “C” shape. C-shaped member is attached to an x-ray source 114 and an image receptor 116. The space within C-arm 104 of the arm provides room for the physician to attend to the patient substantially free of interference from the x-ray support structure.

The C-arm is mounted to enable rotational movement of the arm in two degrees of freedom, (i.e. about two perpendicular axes in a spherical motion). C-arm is slidably mounted to an x-ray support structure, which allows orbiting rotational movement of the C-arm about its center of curvature, which may permit selective orientation of x-ray source 114 and image receptor 116 vertically and/or horizontally. The C-arm may also be laterally rotatable, (i.e. in a perpendicular direction relative to the orbiting direction to enable selectively adjustable positioning of x-ray source 114 and image receptor 116 relative to both the width and length of the patient). Spherically rotational aspects of the C-arm apparatus allow physicians to take x-rays of the patient at an optimal angle as determined with respect to the particular anatomical condition being imaged.

The O-arm® 106 illustrated in FIG. 11 includes a gantry housing 124 which may enclose an image capturing portion, not illustrated. The image capturing portion includes an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of the patient to be acquired from multiple directions or in multiple planes.

The O-arm® 106 with the gantry housing 124 has a central opening for positioning around an object to be imaged, a source of radiation that is rotatable around the interior of gantry housing 124, which may be adapted to project radiation from a plurality of different projection angles. A detector system is adapted to detect the radiation at each projection angle to acquire object images from multiple projection planes in a quasi-simultaneous manner. The gantry may be attached to a support structure O-arm® support structure, such as a wheeled mobile cart with wheels, in a cantilevered fashion. A positioning unit translates and/or tilts the gantry to a planned position and orientation, preferably under control of a computerized motion control system. The gantry may include a source and detector disposed opposite one another on the gantry. The source and detector may be secured to a motorized rotor, which may rotate the source and detector around the interior of the gantry in coordination with one another. The source may be pulsed at multiple positions and orientations over a partial and/or full three hundred and sixty degree rotation for multi-planar imaging of a targeted object located inside the gantry. The gantry may further comprise a rail and bearing system for guiding the rotor as it rotates, which may carry the source and detector. Both and/or either O-arm® 106 and C-arm 104 may be used as automated imaging system to scan a patient and send information to the surgical system 2.

Images captured by an imaging system can be displayed on the XR headset 920 and/or another display device of the computer platform 910, the surgical robot 4, and/or another component of the surgical system 900. The XR headset 920 may be connected to one or more of the imaging devices 104 and/or 106 and/or to the image database 950, e.g., via the computer platform 910, to display images therefrom. A user may provide control inputs through the XR headset 920, e.g., gesture and/or voice based commands, to control operation of one or more of the imaging devices 104 and/or 106 and/or the image database 950.

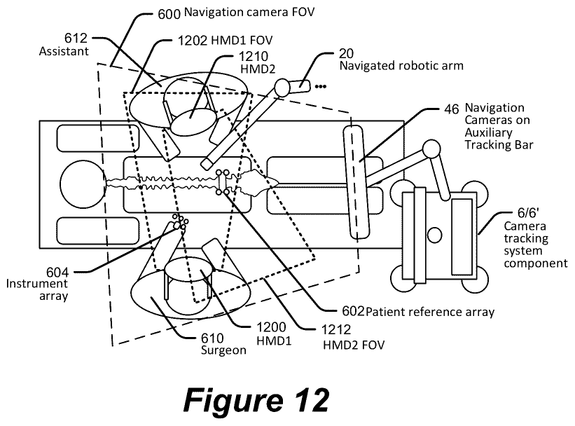

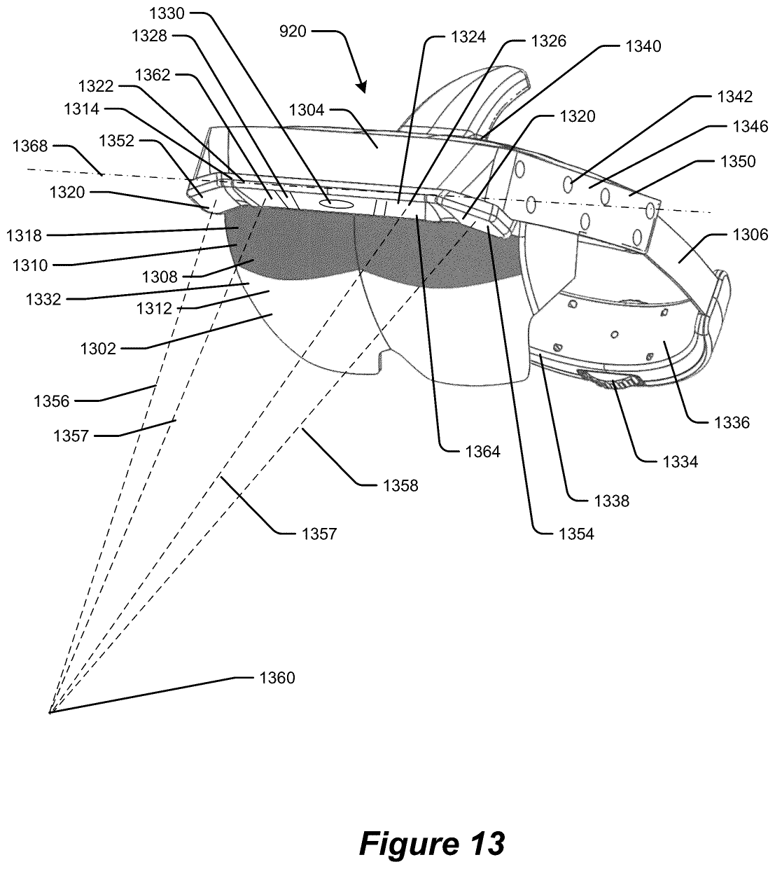

FIG. 12 illustrates a block diagram view of the components of a surgical system that include a pair of XR headsets 1200 and 1210 (head-mounted displays HMD1 and HMD2), which may correspond to the XR headset 920 shown in FIG. 13 and operate in accordance with some embodiments of the present disclosure.

Referring to the example scenario of FIG. 12, the assistant 612 and surgeon 610 are both wearing the XR headsets 1210 and 1210, respectively. It is optional for the assistant 612 to wear the XR headset 1210. The XR headsets 1200 and 1210 are configured to provide an interactive environment through which the wearers can view and interact with information related to a surgical procedure as will be described further below. This interactive XR based environment may eliminate a need for the tech personnel 614 to be present in the operating room and may eliminate a need for use of the display 34 shown in FIG. 6. Each XR headset 1200 and 1210 can include one or more cameras that are be configured to provide an additional source of tracking of DRAs or other reference arrays attached to instruments, an anatomical structure, the end effector 26, and/or other equipment. In the example of FIG. 12, XR headset 1200 has a field-of-view (FOV) 1202 for tracking DRAs and other objects, XR headset 1210 has a FOV 1212 partially overlapping FOV 1202 for tracking DRAs and other objects, and the navigation cameras 46 has another FOV 600 partially overlapping FOVs 1202 and 1212 for tracking DRAs and other objects.

If one or more cameras is obstructed from viewing a DRA attached to a tracked object, e.g., a surgical instrument, but the DRA is in view of one or more other cameras the tracking subsystem 830 and/or navigation controller 828 can continue to track the object seamlessly without loss of navigation. Additionally, if there is partial occlusion of the DRA from the perspective of one camera, but the entire DRA is visible via multiple camera sources, the tracking inputs of the cameras can be merged to continue navigation of the DRA. One of the XR headsets and/or the navigation cameras 46 may view and track the DRA on another one of the XR headsets to enable the computer platform 910 (FIGS. 9 and 14), the tracking subsystem 830, and/or another computing component to determine the pose of the DRA relative to one or more defined coordinate systems, e.g., of the XR headsets 1200/1210, the navigation cameras 46, and/or another coordinate system defined for the patient, table, and/or room.

The XR headsets 1200 and 1210 can be operatively connected to view video, pictures, and/or other information received from and/or to provide commands that control various equipment in the surgical room, including but not limited to neuromonitoring, microscopes, video cameras, and anesthesia systems. Data from the various equipment may be processed and displayed within the headset, for example the display of patient vitals or the microscope feed.

### Example XR Headset Components and Integration to Navigated Surgery, Surgical Robots, and Other Equipment

FIG. 13 illustrates an XR headset 920 which is configured in accordance with some embodiments of the present disclosure. The XR headset includes a headband 1306 configured to secure the XR headset to a wearer's head, an electronic component enclosure 1304 supported by the headband 1306, and a display screen 1302 that extends laterally across and downward from the electronic component enclosure 1304. The display screen 1302 may be a see-through LCD display device or a semi-reflective lens that reflects images projected by a display device toward the wearer's eyes.

The display screen 1302 operates as a see-through display screen, also referred to as a combiner, that reflects light from display panels of a display device toward the user's eyes. The display panels can be located between the electronic component enclosure and the user's head, and angled to project virtual content toward the display screen 1302 for reflection toward the user's eyes. The display screen 1302 is semi-transparent and semi-reflective allowing the user to see reflected virtual content superimposed on the user's view of a real-world scene. The display screen 1302 may have different opacity regions, such as the illustrated upper laterally band which has a higher opacity than the lower laterally band. Opacity of the display screen 1302 may be electronically controlled to regulate how much light from the real-world scene passes through to the user's eyes. A high opacity configuration of the display screen 1302 results in high-contrast virtual images overlaid on a dim view of the real-world scene. A low opacity configuration of the display screen 1302 can result in more faint virtual images overlaid on a clearer view of the real-world scene. The opacity may be controlled by applying an opaque material on a surface of the display screen 1302.

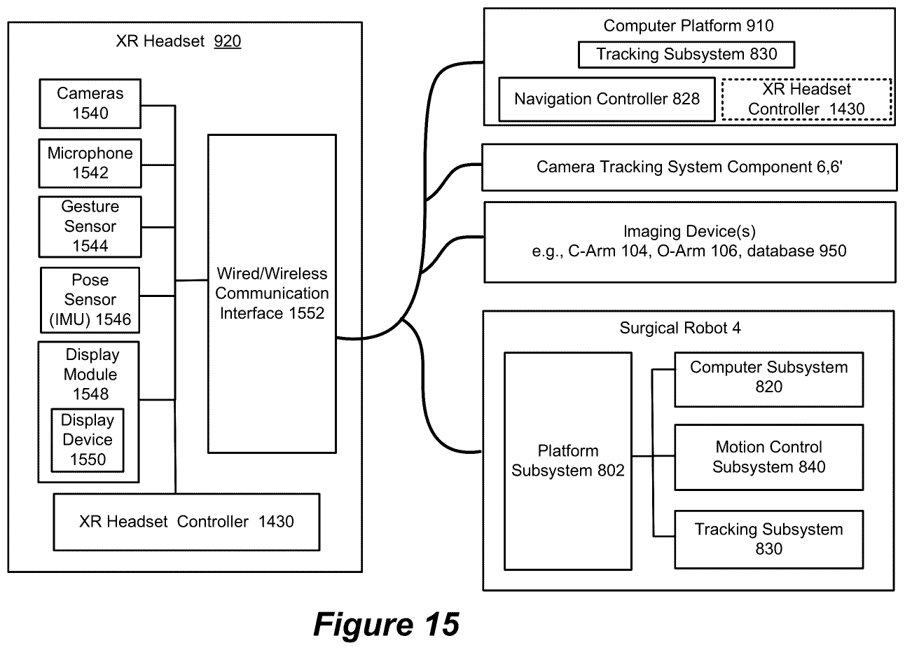

According to some embodiments the surgical system includes an XR headset 920 and an XR headset controller, e.g., controller 1430 in FIG. 14. The XR headset 920 is configured to be worn by a user during a surgical procedure and has a see-through display screen 1302 that is configured to display an XR image and to allow at least a portion of a real-world scene to pass therethrough for viewing by the user. The XR headset 920 also includes an opacity filter positioned between at least one of the user's eyes and the real-world scene when the see-through display screen 1302 is viewed by the user. The opacity filter is configured to provide opaqueness to light from the real-world scene. The XR headset controller is configured to communicate with a navigation controller, e.g., controller(s) 828A, 828B, and/or 828C in FIG. 15, to receive navigation information from the navigation controller which provides guidance to the user during the surgical procedure on an anatomical structure, and is further configured to generate the XR image based on the navigation information for display on the see-through display screen 1302.

Opacity of the display screen 1302 may be configured as a gradient having a more continuously changing opacity with distance downward from a top portion of the display screen 1302. The gradient's darkest point can be located at the top portion of the display screen 1302, and gradually becoming less opaque further down on the display screen 1302 until the opacity is transparent or not present. In an example further embodiment, the gradient can change from about 90% opacity to entirely transparent approximately at the mid-eye level of the display screen 1302. With the headset properly calibrated and positioned, the mid-eye level can correspond to the point where the user would look straight out, and the end of the gradient would be located at the “horizon” line of the eye. The darker portion of the gradient will allow crisp, clear visuals of the virtual content and help to block the intrusive brightness of the overhead operating room lights.

Variations in opacity can be achieved using an opacity filter 1308, which may be integrated into the display screen 1302 or which may be a separate component that is configured to be positioned in the field of view of a user wearing the XR headset 920. Using an opacity filter 1308 in this manner enables the XR headset 920 to provide VR capabilities, by substantially or entirely blocking light from the real-world scene, along an upper portion of the display screen 1302 and to provide AR capabilities along a middle or lower portion of the display screen 1302. This allows the user to have the semi-translucence of AR where needed and allowing clear optics of the patient anatomy during procedures. Configuring the opacity filter 1308 as a gradient instead of as a more constant opacity band can enable the wearer to experience a more natural transition between a more VR type view to a more AR type view without experiencing abrupt changes in brightness of the real-world scene and depth of view that may otherwise strain the eyes such as during more rapid shifting between upward and downward views.

The display panels and display screen 1302 can be configured to provide a wide field of view see-through XR display system. In one example configuration they provide an 80° diagonal field-of-view (FOV) with 55° of vertical coverage for a user to view virtual content. Other diagonal FOV angles and vertical coverage angles can be provided through different size display panels, different curvature lens, and/or different distances and angular orientations between the display panels and curved display screen 1302.

As further shown in FIG. 13, an opacity filter 1308 can be configured as laterally extending bands 1310, 1312 having different light transmissivities. In some embodiments, the XR headset controller is configured to display in a region of the see-through display screen aligned with a first laterally extending band 1310 of the opacity filter 1308 at least one of: 2D Axial, Sagittal, and/or Coronal view images of patient anatomy; a planned and/or currently tracked surgical tool pose; graphical model of surgical implant location; video from a medical instrument; and user selectable menu items triggering operations controlling medical equipment. The XR headset controller is further configured to display in another region of the see-through display screen that is aligned with a second laterally extending band 1312 of the opacity filter 1308 at least one of: a 3D graphical model of the anatomical structure and surgical planning information; 3D graphical model of a surgical instrument; animated 3D graphical model of a surgical instrument displayed with a pose relative to a graphical model of the anatomical structure that is modified to track in real-time measured poses of the surgical instrument relative to the anatomical structure; and a graphical model of the anatomical structure and the navigation information from the navigation controller which provides visual guidance to the user during the surgical procedure on the anatomical structure. In an alternate embodiment, the opacity filter 1308 may include a single laterally extending band 1310, with the XR headset controller displaying content in another region of the see-through display screen 1302 that is not aligned with the laterally extending band 1310 of the opacity filter 1308.

It is noted that while an unobstructed view of a prone patient is easily obtained by looking downwards with both the eyes and head, an augmented view of the patient can also be obtained by pitching the head down a bit further to look through the middle lens region.

In this manner the XR headset can be configured to provide the mixed capabilities and benefits of both VR and AR, straddling the VR-AR continuum in such a way as to maximize the utility for applications such as live interoperative surgery. The XR headset provides options for how much contrast is to be provided between displayed AR images (virtual content such as medical imagery) and the real-world scene, by selectively displaying the AR images within the high opacity upper lens region or in the lower opacity middle lens region. The user can make subtle head pitching movement to adjust the various regions relative to the real-world scene, e.g., to obtain an AR image overlay on an anatomical structure and to alternatively obtain an unobstructed view of the anatomical structure. The XR headset 920 can be configured to identify hand gestures and/or voice commands that control what types of AR content is displayed where on the lens forming the see-through display screen.

In this example, the display screen 1302 includes an optical combiner 1318 having an 80° diagonal field of view with a vertical field of view larger than many conventional wide-aspect-ratio designs, which provides many advantages in surgical and other environments. For example, smaller fields of view may force the user to move their head around in order to see significant amount of content, which is not ideal in a surgical setting where surgeon fatigue is known to impact outcomes. The large combiner 1318 can also act as a blood splatter guard while allowing the wearer to wear their own prescription glasses (or primary eye protection) underneath the display 1302.

In this embodiment, the XR headset 920 of FIG. 13 further includes a plurality of cameras 1314 having different capabilities and functions for providing accurate and precise display of images to a user. The XR headset 920 includes a frame 1350 configured to be worn by a user's head.

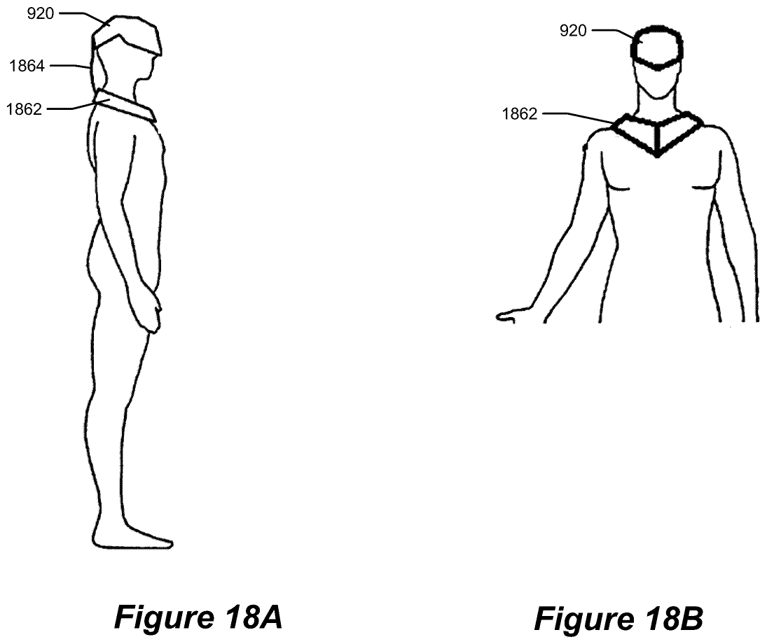

A stereo camera tracking subassembly 1316 is provided for tracking surgical instruments, references, robotic end effectors, etc., with accuracy and ergonomic benefits that are not available with conventional general purpose cameras and lenses. The stereo camera tracking subassembly 1316 includes a pair of high-precision stereo cameras 1320. The stereo visible light cameras 1320 are angled approximately 6° with respect to a horizontal of the tracking subassembly 1316 (i.e., 12° with respect to each other). This is desirable for accurate tracking of surgical instruments, robotic end effectors and patient references during surgery because the fields of view of the stereo visible light cameras 1320 converge at the typical distance for the presence of such instruments during surgery. The field of view of the stereo visible light cameras 1320 is selected to be under 90° in this embodiment for low distortion and high accuracy, while still allowing of an appropriately large stereo tracking frustum as shown in FIGS. 18A-18D for example.

In this embodiment, the stereo visible light cameras 1320 track in visible light. This allows the cameras to see tracking markers, the patient, and everything that is going on in the visible surgical field. In this example, the stereo visible light cameras 1320 do not rely on near infrared (NIR) or other non-visible illumination. The omission of NIR illuminators in this embodiment significantly reduces power consumption, weight, and component cost for the stereo visible light cameras 1320, and may improve the appearance of the headset 920.

In this example, the stereo visible light cameras 1320 are mechanically connected via a rigid mounting element 1322, e.g., a single carbon fiber tube. The material of the carbon fiber tube 1322 is very rigid and is relatively insusceptible to thermal expansion, e.g., due to internal heating of the headset 920. This rigidity and thermal stability in turn allows for the tracking subassembly 1316 to better maintain calibration and accuracy. In this example, the stereo visible light cameras 1320 include a right-side visible light camera 1352 and a left-side visible light camera 1354 coupled to the carbon fiber tube 1322 on opposite sides of the carbon fiber tube 1322. The right-side visible light camera 1352 defines a field of view having a first center in a first direction 1356 extending away from the frame 1350, and the left-side visible light camera 1354 defines a field of view having a second center in a second direction 1358 extending away from the frame 1350. In this example, the first direction 1356 and the second direction 1358 intersect at an intersection point 1360 within a field of view of the user when the user is wearing the frame 1350, and define an angle that is greater than 10 degrees and smaller than 15 degrees (e.g. substantially 12 degrees in this embodiment). In this example, the frame 1350 also defines a substantially horizontal reference plane 1368 when the frame 1350 is being worn by the user, with the first direction 1356 and the second direction 1358 being angled downwardly with respect to the horizontal reference plane 1368 at a downward angle that is greater than 50 degrees and less than 60 degrees (e.g., substantially 55 degrees in this embodiment).

The headset 920 further includes an NIR camera tracking subassembly 1324 that includes stereo NIR cameras 1326 with a much larger (e.g., 180°) field of view, and that substantially includes the stereo visible light cameras 1320 field of view. In this example, the stereo NIR cameras 1326 may have a lower resolution than the stereo visible light cameras 1320. Each of the stereo NIR cameras 1326 has at least one NIR LED 1328, which can be desynchronized from other tracking apparatuses in the OR as desired, so as not to interfere with such apparatuses, for example. This allows the NIR camera tracking subassembly 1324 to track hand movement of the surgeon independently of the surgical tool tracking of the visible light tracking subassembly 1316. Because the LEDs 1328 have a relatively low intensity, objects in the field of view that are closer to the LEDs 1328, such as a surgeon's hands, are brightly illuminated while objects that are farther away, such as background objects, are dimly illuminated. This allows a surgeon's hands to be brightly illuminated, with high contrast, by the NIR LEDs 1328, which in turn allows for more accurate hand tracking by the NIR camera tracking subassembly 1324.

In this example, the stereo NIR cameras 1326 include a right-side NIR camera 1362 and a left-side NIR camera 1364 coupled on opposite sides of the carbon fiber tube 1322. The stereo NIR cameras 1326 also include at least one NIR light-emitting diode (LED) 1366 configured to illuminate a region within a field of view of the stereo NIR cameras 1326. In this example the stereo visible light cameras 1320 and the stereo NIR cameras 1326 are both configured to capture respective stereoscopic visible light and NIR images within a common field of view of the user when the user is wearing the frame 1350. In this example, the stereo visible light cameras 1320 are configured to capture the stereoscopic visible light images at a first resolution, and the stereo NIR cameras 1326 are configured to capture the stereoscopic NIR images at a second image resolution less than the first resolution. In this example, the stereo NIR cameras 1326 each define respective fields of view having centers that extend in directions 1357 extending away from the frame 1350. In this example, the directions 1357 of the stereo NIR cameras 1326 intersect at the same intersection point 1360 within a field of view of the user as the stereo visible light cameras 1320, but it should be understood that the directions 1357 of the stereo NIR cameras 1326 may converge on a different intersection point, as desired.

In some examples, the separation between the stereo NIR cameras 1326 may be less than or equal to 96 millimeters, or may be less than or equal to 64 millimeters. as desired. In this example, the stereo NIR cameras 1326 are separated by approximately 96 millimeters so that the lens 1332 of the loupe camera 1330 (described below) does not interfere with the stereo NIR cameras 1326 or LEDs 1328. In this example, the headset 920 may also include a 6-axis inertial measurement unit (IMU) (not shown) that may include a 3-axis gyroscope and a 3-axis accelerometer. This IMU may allow for 1 kHz low latency head tracking, and may also infer the pitch and roll of external objects relative to a known coordinate system (i.e., gravity). This permits the NIR camera tracking subassembly 1324 to allow for interaction with virtual (i.e., software) content in a virtual space without breaking sterility. In this manner, the headset 920 may capture visible light stereoscopic images and NIR stereoscopic images of a scene during a particular time period. Locations of different objects within a three dimensional space may then be determined for the different sets of images.

The headset 920 further includes a centrally located loupe camera 1330 having high resolution (e.g., greater than the resolution of the stereo visible light cameras) and color capability, with a relatively low (e.g., 25°) field of view lens 1332. This loupe camera 1330 may provide a digital zoom feature, which is up to 5× magnification in this embodiment. It should also be understood that an optical zoom mechanism may also be used. However, in this embodiment, a digital zoom is used to reduce cost, weight, and mechanical complexity. This loupe camera 1330 may be used in conjunction with the NIR camera tracking subassembly 1324, for example to use hand tracking to pinch and drag digital imagery in and out of the field of view of the loupe camera 1330, with color content dynamically zooming and panning on request. In this manner, the need for traditional analog surgical loupes, which require additional heavy and distracting eyewear, is reduced or eliminated.

In this example, all of the headset camera apparatuses, i.e., the stereo cameras 1320, 1326, and/or loupe camera 1330, may be angled downwardly. For example, in this embodiment, the stereo visible light cameras 1320 may be angled downwardly at an approximate downward angle of 55°. The stereo NIR cameras 1326 may be angled downwardly with respect to the horizontal reference plane 1368 at a downward angle that is between 15° and 45° (e.g., substantially 35° in this embodiment). Applicants have recognized through experimentation that this approximate downward angle is advantageous for typical surgical procedures where the patient laying prone on a bed above waist level of the surgeon.

The headset 920 further includes a tightening wheel 1334 that allows a rear headset strap 1336 to be adjusted quickly and comfortably with one hand. The wheel 1334 in this example is made of a metal, such as steel, and may be heavier than necessary, to allow the wheel 1334 to act as a weighted counterbalance to balance the headset 920 on the user's head. Foams 1338 and/or other comfort elements may be removable for cleaning, sterilization, and/or replacement, and can be customized for different users and/or preferences. In this example, a top portion 1340 curves back over the head in such a way that the headset 920 is well supported without the need for a strap connecting the top portion 1340 to the rear headset strap 1336, thereby reducing weight and enhancing comfort for the user's head.

The headset 920 may include tracking markers 1342 that may by external cameras for example, for collocating the headset 920 relative to patient anatomy and surgical equipment. In this example, the tracking markers 1342 are part of sticker plates 1346 that may be mounted into recessed and/or keyed locations for increased accuracy. The sticker plates 1346 may feature different patterns in order to differentiate one headset 920 from another or to recognize the left side of the headset from the right, for example.

Referring now to FIG. 14, an alternative headset 1420 is illustrated having additional features. In this regard, headset 1420 includes a connector port 1450 for attachment of a removable surgical head lamp (not shown) or other electronic device or accessory. The ability to remove this lamp and/or other accessory allows the weight of the headset 1420 to be reduced for users that do not require an accessory, and also allows users to select from different kinds of lamps and other accessories for different applications. In this example, the connector port includes one or more keyed recesses 1452 for removably retaining a complementary keyed protrusion of the removable accessory, and one or more electrical contacts for providing power to the accessory and/or for data transfer between the accessory and the headset 1430.

The headset 1420 further includes a plurality of cooling vents 1454 to facilitate airflow through the headset 1420 and around the internal electronic components thereof, to prevent overheating. In this example, inlet cooling vents (not shown) may be positioned under the headset and behind the display screen 1402. This has the advantage of preventing blood spatter or other fluids or debris from being drawn into the inlet cooling vents during use. An internal fan (not shown) draws air through the headset 1420 and expels the hot air through the cooling vents 1454 on top of the headset 1420. In this example, the cooling vents 1454 may include multiple staggered layers, to further reduce ingress of liquids while facilitating airflow. In this example at least one of the layers may be formed from carbon fiber, to further strengthen and stiffen the headset 1420 while reducing its weight. It should understood, however, that other cooling vent arrangements may be used, and may be positioned with different geometries and airflow patterns, as desired.

FIG. 15 illustrates electrical components of the XR headset 920 that can be operatively connected to the computer platform 910, to one or more of the imaging devices, such as the C-arm imaging device 104, the O-arm imaging device 106, and/or the image database 950, and/or to the surgical robot 800 in accordance with various embodiments of the present disclosure.

The XR headset 920 provides an improved human interface for performing navigated surgical procedures. The XR headset 920 can be configured to provide functionalities, e.g., via the computer platform 910, that include without limitation any one or more of: identification of hand gesture based commands and/or voice based commands, display XR graphical objects on a display device 1550. The display device 1550 may a video projector, flat panel display, etc., which projects the displayed XR graphical objects on the display screen 1302. The user can view the XR graphical objects as an overlay anchored to particular real-world objects viewed through the display screen 1302 (FIG. 13). The XR headset 920 may additionally or alternatively be configured to display on the display device 1550 video feeds from cameras mounted to one or more XR headsets 920 and other cameras.

Electrical components of the XR headset 920 can include a plurality of cameras 1540, a microphone 1542, a gesture sensor 1544, a pose sensor (e.g., inertial measurement unit (IMU)) 1546, a display module 1548 containing the display device 1550, and a wireless/wired communication interface 1552. As will be explained below, the cameras 1540 of the XR headset may be visible light capturing cameras, near infrared capturing cameras, or a combination of both.

The cameras 1540 may be configured operate as the gesture sensor 1544 by capturing for identification user hand gestures performed within the field of view of the camera(s) 1540. Alternatively the gesture sensor 1544 may be a proximity sensor and/or a touch sensor that senses hand gestures performed proximately to the gesture sensor 1544 and/or senses physical contact, e.g. tapping on the sensor or the enclosure 1304. The pose sensor 1546, e.g., IMU, may include a multi-axis accelerometer, a tilt sensor, and/or another sensor that can sense rotation and/or acceleration of the XR headset 920 along one or more defined coordinate axes. Some or all of these electrical components may be contained in the component enclosure 1304 or may be contained in another enclosure configured to be worn elsewhere, such as on the hip or shoulder.

As explained above, the surgical system 2 includes a camera tracking system component 6/6′ and a tracking subsystem 830 which may be part of the computer platform 910. The surgical system may include imaging devices (e.g., C-arm 104, O-arm 106, and/or image database 950) and/or a surgical robot 4. The tracking subsystem 830 is configured to determine a pose of DRAs attached to an anatomical structure, an end effector, a surgical tool, etc. A navigation controller 828 is configured to determine a target pose for the surgical tool relative to an anatomical structure based on a surgical plan, e.g., from a surgical planning function performed by the computer platform 910 of FIG. 9, defining where a surgical procedure is to be performed using the surgical tool on the anatomical structure and based on a pose of the anatomical structure determined by the tracking subsystem 830. The navigation controller 828 may be further configured to generate steering information based on the target pose for the surgical tool, the pose of the anatomical structure, and the pose of the surgical tool and/or the end effector, where the steering information indicates where the surgical tool and/or the end effector of a surgical robot should be moved to perform the surgical plan.

The electrical components of the XR headset 920 can be operatively connected to the electrical components of the computer platform 910 through a wired/wireless interface 1552. The electrical components of the XR headset 920 may be operatively connected, e.g., through the computer platform 910 or directly connected, to various imaging devices, e.g., the C-arm imaging device 104, the I/O-arm imaging device 106, the image database 950, and/or to other medical equipment through the wired/wireless interface 1552.

The surgical system 2 further includes at least one XR headset controller 1430 (also referred to as “XR headset controller” for brevity) that may reside in the XR headset 920, the computer platform 910, and/or in another system component connected via wired cables and/or wireless communication links. Various functionality is provided by software executed by the XR headset controller 1430. The XR headset controller 1430 is configured to receive navigation information from the navigation controller 828 which provides guidance to the user during the surgical procedure on an anatomical structure, and is configured to generate an XR image based on the navigation information for display on the display device 1550 for projection on the see-through display screen 1302.

The configuration of the display device 1550 relative to the display screen (also referred to as “see-through display screen”) 1302 is configured to display XR images in a manner such that when the user wearing the XR headset 920 looks through the display screen 1302 the XR images appear to be in the real world. The display screen 1302 can be positioned by the headband 1306 in front of the user's eyes.

The XR headset controller 1430 can be within a housing that is configured to be worn on a user's head or elsewhere on the user's body while viewing the display screen 1302 or may be remotely located from the user viewing the display screen 1302 while being communicatively connected to the display screen 1302. The XR headset controller 1430 can be configured to operationally process signaling from the cameras 1540, the microphone 1542, and/or the pose sensor 1546, and is connected to display XR images on the display device 1550 for user viewing on the display screen 1302. Thus, the XR headset controller 1430 illustrated as a circuit block within the XR headset 920 is to be understood as being operationally connected to other illustrated components of the XR headset 920 but not necessarily residing within a common housing (e.g., the electronic component enclosure 1304 of FIG. 13) or being otherwise transportable by the user. For example, the XR headset controller 1430 may reside within the computer platform 910 which, in turn, may reside within a housing of the computer tracking system component 6′ shown in FIGS. 3B and 3C.

### Example XR Headset Component Optical Arrangement

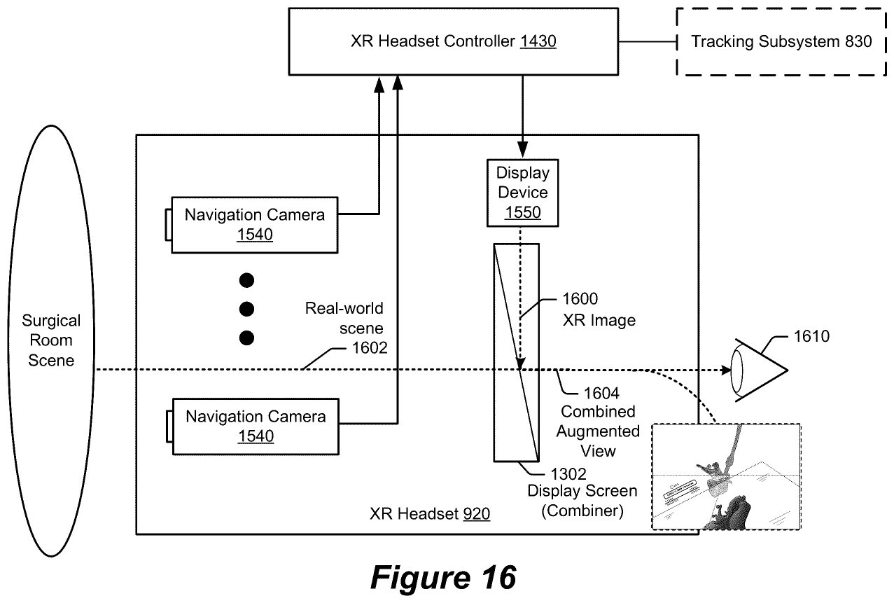

FIG. 16 illustrates a block diagram showing an arrangement of optical components of the XR headset 920 in accordance with some embodiments of the present disclosure. Referring to FIG. 16, the display device 1550 is configured to display XR images 1600 generated by the XR headset controller 1430, light from which is projected by the display device 1550 as XR images 1600 toward the display screen 1302. The display screen 1302 is configured to combine light of the XR images 1600 and light from the real-world scene 1602 into a combined augmented view 1604 that is directed to the user's eye(s) 1610. The display screen 1302 configured in this manner operates as a see-through display screen. The XR headset 920 can include any plural number of navigation cameras 1540. The cameras 1540 may be visible light capturing cameras, near infrared capturing cameras, or a combination of both.

The XR headset operations can display both 2D images and 3D models on the display screen 1302. The 2D images may preferably be displayed in a more opaque band of the display screen 1302 (upper band) and the 3D model may be more preferably displayed in the more transparent band of the display screen 1302, otherwise known as the environmental region (bottom band). Below the lower band where the display screen 1302 ends the wearer has an unobstructed view of the surgical room. It is noted that where XR content is display on the display screen 1302 may be fluidic. It is possible that where the 3D content is displayed moves to the opaque band depending on the position of the headset relative to the content, and where 2D content is displayed can be placed in the transparent band and stabilized to the real world. Additionally, the entire display screen 1302 may be darkened under electronic control to convert the headset into virtual reality for surgical planning or completely transparent during the medical procedure. As explained above, the XR headset 920 and associated

Other types of XR images (virtual content) that can be displayed on the display screen 1302 can include, but are not limited to any one or more of:
1) 2D Axial, Sagittal and/or Coronal views of patient anatomy;
2) overlay of planned vs currently tracked tool and surgical implant locations;
3) gallery of preoperative images;
4) video feeds from microscopes and other similar systems or remote video conferencing;
5) options and configuration settings and buttons;
6) floating 3D models of patient anatomy with surgical planning information;
7) real-time tracking of surgical instruments relative to floating patient anatomy;
8) augmented overlay of patient anatomy with instructions and guidance; and
9) augmented overlay of surgical equipment.

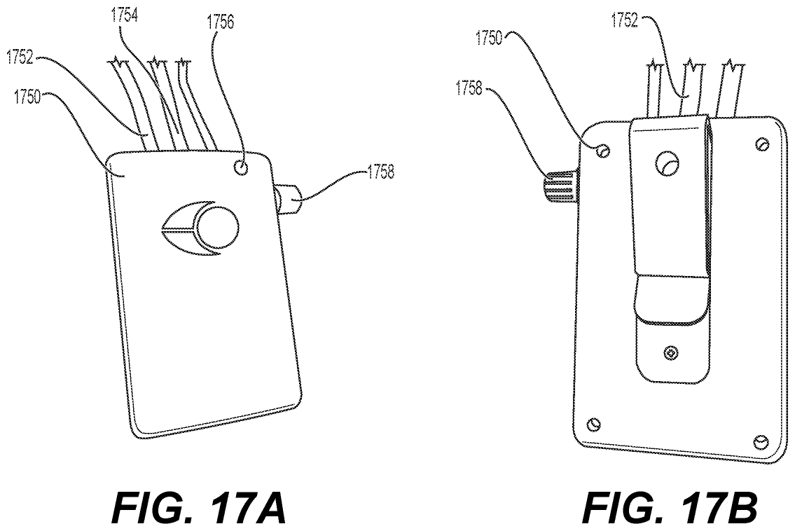

Referring now to FIGS. 17A and 17B, a tether pack 1750 is connected between the headset 920 and computer platform 910. The tether pack 1750 may provide power and data transfer for the headset 920 and/or any accessories, such as lamps via a cable apparatus 1752. The cable apparatus 1752 may be detachable or permanently connected, as desired. The tether pack may include a quick-disconnect component 1754 for quick and safe disconnection from the computer platform, for example, if the cable becomes tangled or caught, thereby reducing the risk of neck strain or injury and/or damage to the headset 920 if excessive force is applied to the cable. Connection status may be monitored in this example by an LED indicator 1756, which may indicate whether the headset 920 is powered, communicating properly, and/or passing internal diagnostic tests, etc. An input device 1758, such as a wheel in this example, may control operation of headset components or accessories, such as lamp brightness for example. As shown in FIG. 17B, the tether pack 1750 may be secured to the user, e.g., on a user's belt using a belt clip 1760, to reduce the weight and bulk of the cable with respect to the headset 930 when moving around a surgical or other environment.

Referring now to FIGS. 18A and 18B, additional components may be provided in a tethered collar accessory 1862, to be worn on around a user's neck, and shoulders to further reduce strain on a user's head and neck. In addition to causing neck strain, excess weight in the headset 920 can cause skin chafing, sweating, excess heat generation, and general discomfort. However, it is possible to remove many functional components of the headset 920 from the headset itself while maintaining mobility of the user and headset 920. In this example, the displays, cameras and certain sensors (discussed above), such as inertial and other low-latency sensors, remain on the headset 920. Other components, such as computing devices, batteries, charging connectors, wireless communication components, audio input and output systems, and/or input devices such as buttons, etc., can be included in the collar accessory 1862 and may be connected to components of the headset 920 by a wired tether 1864 for example. It should be understood that this single tether 1864 may be disconnected from one or both ends, as desired, for added flexibility in selecting cable length and for simplified repair of damaged cabling. Likewise, different headsets 920 may be used interchangeably with the collar accessory 1862. The collar accessory 1862 may further reduce headset cable length and, in some embodiments, could provide a docking mechanism the headset 920. It should also be understood that the relatively short length and location of the tether 1864 may also reduce snag hazards while improving signal integrity.

Another benefit of using a separate accessory is that the accessory can be detachable, so that components may be interchangeable and easily replaceable. In this manner, damaged components can be more easily replaced, and different accessories may be swapped out with different components having different functionalities.

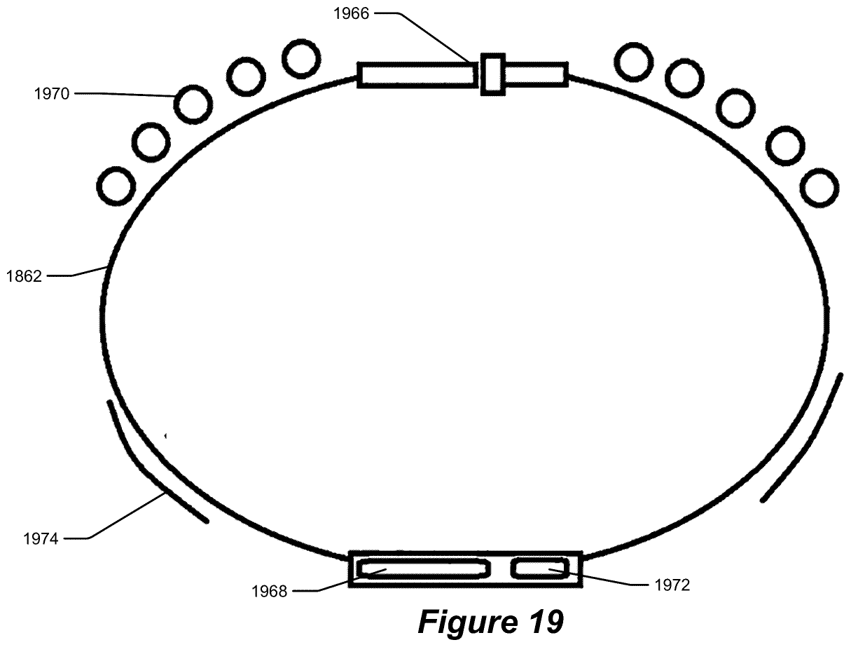

The collar accessory 1862 of FIGS. 18A-18B may be worn inside clothing, such as surgical scrubs. Referring now to FIG. 19, for example, a member of the surgical staff, e.g., a scrub nurse, could place the collar accessory 1862 around the back of a user's neck under the user's sterile scrubs. A magnetic latch 1966 may bind automatically to secure the collar accessory 1862 around the user's neck and shoulders. The collar accessory 1862 can then easily be removed by grabbing the back of the collar accessory 1862 and pulling it free, again without breaking sterility. This functionality may remove the need for a sterile drape or other additional sterility measures during surgery.

In this example, a computer module 1968 is positioned at the rear of the collar accessory 1862 and batteries 1970 are positioned towards the front of the collar accessory 1862 on either side of the magnetic latch 1966. This helps balance the weight distribution of the collar accessory 1862, thereby reducing the risk of the collar accessory 1862 falling off during use. The computer module 1968 may include a power and data connector 1972 for removably tethering the collar accessory 1862 and headset 920 to a computer platform 910 for example. Audio systems 1974, such as microphones and/or speakers may also be included in the collar accessory 1862. The speakers may employ directional audio, or may be incorporated in headset/earbud attachments, etc. as desired. For example, speakers may provide adequate audio performance, even when used through surgical scrub material. For embodiments that use headset or earbuds, the proximity of the collar accessory 1862 allows for shorter audio cabling, thereby reducing overall weight.

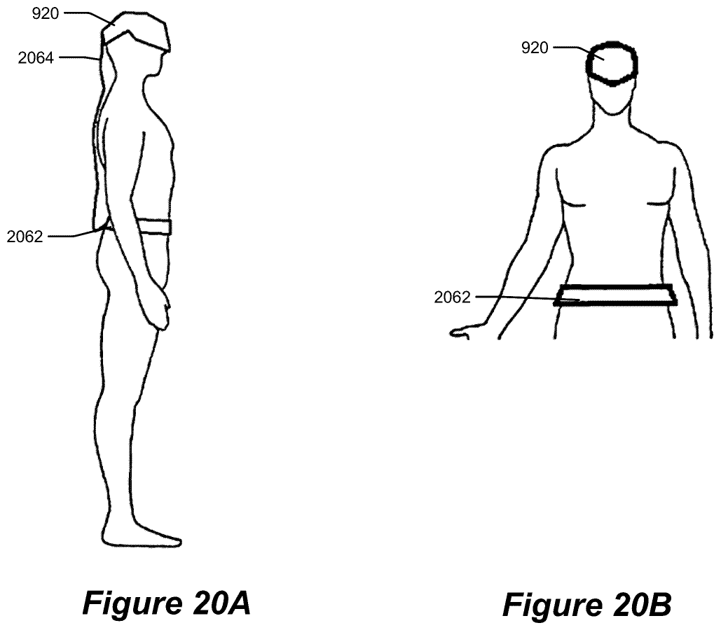

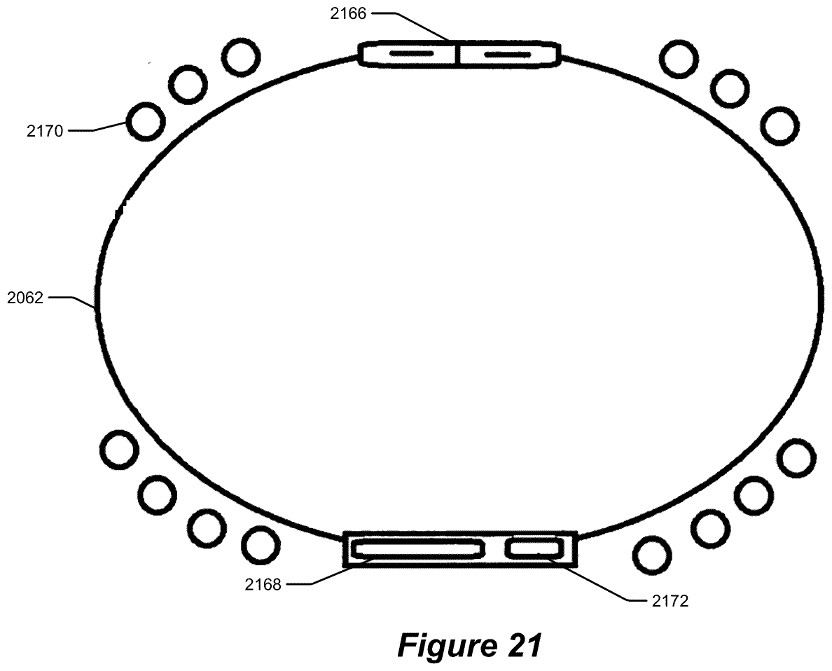

Referring now to FIGS. 20A, 20B, and 21, a belt accessory 2062 is illustrated. In this embodiment, a single cable tether 2064 containing display, data and headset power is connected between the headset 930 and the belt accessory 2062. As shown in FIG. 21 in particular, a belt clip 2166 may be used to secure the belt accessory 2062 around a user's waist. Batteries 2170 are distributed around the belt accessory 2062 to evenly distribute the weight of the components around the user's waist, and to increase ergonomics and comfort for the user. A computer module 2168 and power and data connector 2172 are disposed at a rear of the belt accessory 2062. Similar to the collar accessory 1862, the belt accessory 2062 can be worn under surgical scrubs and/or may be sheathed in a sterile drape, as desired, to maintain sterility while allowing adjustment of the belt accessory 2062 and for external components, such as input devices, LEDs, etc. to be visible and accessible to the user and/or surgical staff.

### Further Definitions and Embodiments:

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

What is claimed is:

1. A head-mounted extended reality (XR) display device comprising:
a frame configured to be worn by a user's head;
a rigid mounting element coupled to the frame;
a camera coupled to the rigid mounting element;
wherein the camera is configured to capture stereoscopic visible light images within a field of view of the user when the user is wearing the frame, and
a tracking system adapted to determine a pose of dynamic reference arrays attached to a patient and to surgical instruments based on stereoscopic visible light images received through the camera, the tracking system further adapted to determine hand gesture based commands based on stereoscopic NIR images received through the camera.

2. The XR display device of claim 1, wherein the camera defines a field of view having a first center in a first direction extending away from the frame,
wherein the camera defines a field of view having a second center in a second direction extending away from the frame, and
wherein the first direction and the second direction intersect at an intersection point within a field of view of the user when the user is wearing the frame.

3. The XR display device of claim 2, wherein the first direction and the second direction define an angle at the intersection point that is greater than 10 degrees and smaller than 15 degrees.

4. The XR display device of claim 3, wherein the angle is substantially 12 degrees.

5. The XR display device of claim 2, wherein the frame defines a substantially horizontal reference plane when the frame is being worn by the user, and
wherein the first direction and the second direction are angled downwardly with respect to the horizontal reference plane at a downward angle that is greater than 50 degrees and less than 60 degrees when the frame is being worn by the user.

6. The XR display device of claim 5, wherein the downward angle is substantially 55 degrees.

7. The XR display device of claim 1, further comprising:
an optical combiner attached to the frame and adapted to pass real world images toward the user's eyes;
a display mounted to the frame so as to project virtual graphical objects on to the optical combiner to be reflected back towards the user's eyes.

8. The XR display device of claim 1, wherein the camera is configured to capture the stereoscopic visible light images at a first resolution, and to capture the stereoscopic NIR images at a second resolution less than the first resolution.

9. The XR display device of claim 8, further comprising a loupe camera coupled to the frame, the loupe camera configured to capture color images within the field of view of the user at a third resolution greater than the first resolution.

10. An extended reality (XR) camera assembly for a head-mounted display device comprising:
a rigid mounting element configured to be coupled to a head-wearable frame;
a visible light camera coupled to the rigid mounting element;
a near-infrared (NIR) camera coupled to the rigid mounting element;
at least one NIR light-emitting diode (LED) configured to illuminate a region within a field of view of the right-side NIR camera and the left-side NIR camera,
wherein the visible light camera is configured to capture stereoscopic visible light images within a field of view of a user when the user is wearing the frame, and
wherein the NIR camera is configured to capture stereoscopic NIR images within the field of view of the user when the user is wearing the frame,
a tracking system adapted to determine a pose of dynamic reference arrays attached to a patient and to surgical instruments based on stereoscopic visible light images received through the visible light camera, the tracking system further adapted to determine hand gesture based commands based on the stereoscopic NIR images received through the NIR cameras.

11. The XR camera assembly of claim 10, wherein the visible light camera defines a field of view having a first center in a first direction extending away from the frame,
wherein the visible light camera defines a field of view having a second center in a second direction extending away from the frame, and
wherein the first direction and the second direction intersect at an intersection point within a field of view of the user when the user is wearing the frame.

12. The XR camera assembly of claim 11, wherein the first direction and the second direction define an angle at the intersection point that is greater than 10 degrees and smaller than 15 degrees.

13. The XR camera assembly of claim 12, wherein the angle is substantially 12 degrees.

14. The XR camera assembly of claim 11, wherein the frame defines a substantially horizontal reference plane when the frame is being worn by the user, and
wherein the first direction and the second direction are angled downwardly with respect to the horizontal reference plane at a downward angle that is greater than 50 degrees and less than 60 degrees when the frame is being worn by the user.

15. The XR camera assembly of claim 14, wherein the downward angle is substantially 55 degrees.

16. The XR display device of claim 10, further comprising:
an optical combiner attached to the frame and adapted to pass real world images toward the user's eyes;
a display mounted to the frame so as to project virtual graphical objects on to the optical combiner to be reflected back towards the user's eyes.

17. The XR camera assembly of claim 10, wherein the visible light camera and the is configured to capture the stereoscopic visible light images at a first resolution, and
wherein the NIR camera is configured to capture the stereoscopic NIR images at a second resolution less than the first resolution.

18. The XR camera assembly of claim 17, further comprising a loupe camera coupled to the frame, the loupe camera configured to capture color images within the field of view of the user at a third resolution greater than the first resolution.

19. A method of generating extended reality (XR) imagery comprising:
capturing a first plurality of visible light stereoscopic images of a scene within a field of view of a visible light cameras coupled to a rigid mounting element during a time period;
capturing a second plurality of near-infrared (NIR) stereoscopic images of a scene within a NIR cameras coupled to the rigid mounting element during the time period;
determining a pose of dynamic reference arrays attached to a patient and to a surgical instrument based on the first plurality of visible light stereoscopic images within a three-dimensional space; and
determining a hand gesture based commands based on the second plurality of NIR stereoscopic images within the three-dimensional space.

20. The method of claim 19, wherein the pair of visible light cameras are disposed at an angle with respect to each other that is greater than 10 degrees and smaller than 15 degrees.
